(19) United States
(12) Patent Application Publication
(10) Pub. No.: US 2024/0218327 A1
(43) Pub. Date: Jul. 4, 2024

(54) METHOD FOR QUICKLY AND EFFICIENTLY DIFFERENTIATING FUNCTIONAL GLIAL CELLS

(71) Applicant: KOREA UNIVERSITY RESEARCH AND BUSINESS FOUNATION, Seoul (KR)

(72) Inventors: Dae Sung KIM, Seoul (KR); Gyu Bum YEON, Seongnam-si (KR)

(73) Assignee: KOREA UNIVERSITY RESEARCH AND BUSINESS FOUNDATION, Seoul (KR)

(21) Appl. No.: 18/044,667

(22) PCT Filed: Sep. 8, 2021
(86) PCT No.: PCT/KR2021/012183

§ 371 Date: Dec. 20, 2023

(30) Foreign Application Priority Data
Sep. 10, 2020 (KR) 10-2020-0116430
Jan. 22, 2021 (KR) 10-2021-0009498

(51) Int. Cl. C12N 5/079; C12N 15/85

(52) U.S. Cl. CPC C12N 5/0622; C12N 15/85; C12N 2501/60; C12N 2506/02; C12N 2506/45

(57) ABSTRACT

A method for differentiating stem cells into astrocytes according to the present invention is characterized in that stem cells are made to differentiate into mature astrocytes, having similar physiological functions to cells isolated from the adult brain, within 2 weeks by using neural precursor cells as starting cells and overexpressing a transcription factor called NFIB. The cell differentiation process can mimic a process of molecular and functional change similar to the differentiation process of normal astrocytes.

### CROSS REFERENCE TO RELATED APPLICATIONS

This application is a National Stage of International Application No. PCT/KR2021/012183 filed Sep. 8, 2021, claiming priority based on Korean Patent Application No. 10-2020-0116430 filed Sep. 10, 2020 and Korean Patent Application No. 10-2021-0009498 filed Jan. 22, 2021.

### TECHNICAL FIELD

The present invention relates to a method for rapidly and efficiently differentiating functional astrocytes.

### BACKGROUND ART

It is known that astrocytes perform various functions to maintain homeostasis in the nervous system, and loss of their normal functions leads to neurodevelopmental diseases or neurodegenerative diseases. Previous studies of the physiological functions of astrocytes in the nervous system and the pathophysiology of astrocytes in various neurological diseases were primarily conducted using cells isolated from rodent brains. However, it is well-established that there are significant differences between rodent astrocytes and human astrocytes. Therefore, the development of a method for obtaining human astrocytes is essential for advancing neuroscience and treatment technologies for neurological diseases.

Moreover, astrocytes that generated from various sources, including stem cells, must possess molecular and cell physiological properties that closely resemble those found in the human brain.

Although methods for differentiating stem cells, particularly pluripotent stem cells, into astrocytes have been researched, developed, and reported, the following problems are presented:

### (1) It Takes Considerable Time to Obtain Astrocytes.

Non-Patent Document 1 (Nat Biotech, 29(6), 528-534) discloses a technique for differentiating human pluripotent stem cells into astrocytes (or precursor cells thereof).). However, the method according to Non-Patent Document 1 has a disadvantage in that it takes at least 180 days (about 26 weeks or 6 months) to obtain astrocytes from human pluripotent stem cells.

The protocol of Non-Patent Document 3 (Nat Biotechnol, 37(3), 267-275) discloses that it takes about 8 weeks to obtain mature astrocytes from neural stem cells (LTNSCs) derived from human pluripotent stem cells through specific gene introduction (NFIA). As in the protocol of Non-Patent Document 3, where it typically takes 8 to 26 weeks to produce mature astrocytes, it becomes crucial to expedite the differentiation process to ensure the desired quantity of cells can be acquired. This is particularly vital, as each experiment requires a preparation period of at least two months or more.

### (2) The Potential Presence of Non-Neural Cells.

Non-Patent Document 2 (Nat Methods, 15(9), 693-696) is a document that discloses a technique for differentiating astrocytes from human pluripotent stem cells by introducing specific genes (SOX9 and/or NFIB). However, when inducing differentiation from pluripotent stem cells, it may not always be possible to ensure the differentiation of high-purity astrocytes. This is because the differentiation process can sometimes result in a mixed population of astrocytes with undesired cells from other lineages, such as endodermal and mesodermal cells, among others.

(3) Significant Dissimilarity Compared to Astrocytes Isolated from the Human Brain.

Astrocytes generated using the methods outlined in Non-Patent Documents 1 to 3 are presumed to possess a certain level of fundamental physiological functionality consistent with that of astrocytes. However, there is insufficient evidence to ascertain whether cells produced by these methods exhibit a comparable degree of similarity or dissimilarity in the functionality of astrocytes obtained from the human brain tissue. Consequently, there remains a need for a technique that can yield cells with functional characteristics closely mirroring those of astrocytes from human brain tissue within a shorter timeframe.

### DISCLOSURE

### Technical Problem

The present inventors have confirmed that by utilizing neural precursor cells as the initial cell source and overexpressing NFIB, astrocytes can be efficiently differentiated within a short timeframe, thereby completing the present invention.

Accordingly, an object of the present invention is to provide a method for the efficient differentiation of astrocytes, possessing functions akin to cells found in human brain tissue, from neural precursor cells within a brief timeframe.

### Technical Solution

As a means for solving the above problem, the present invention relates to a composition for inducing differentiation from neural precursor cells into astrocytes including: an NFIB protein; or a gene encoding the same, and a method for inducing differentiation.

Hereinafter, the configuration of the present invention will be described in detail.

The present invention provides a composition for inducing the differentiation of neural precursor cells into astrocytes, which includes an NFIB protein or the gene encoding the same.

The nuclear factor IB (NFIB) is known to function as a CCAAT-binding transcription factor for promoters belonging to the CTF/NF-I family. In the present invention, the NFIB may be introduced into neural precursor cells to upregulate the endogenous expression of SOX9 within these cells.

In an exemplary embodiment, the NFIB gene and protein are derived from mammals such as mice, rats, apes, or humans, and may be derived from humans. The full-length of the NFIB gene and protein or a fragment thereof can be used as long as it achieves the effect of the present invention, and the full-length sequence may be GenBank Accession No. NC_000009.12, NC_000070.7, NC_005104.4 or NC_041768.1, but is not limited thereto.

In the present invention, the neural precursor cells may be derived from pluripotent stem cells.

As used herein, the term “pluripotent stem cell (hereinafter referred to as PSC, and in the case of a human, human PSC or hPSC)” denotes a type of stem cell capable of differentiating into any cell type that comprises the body. Pluripotent stem cells encompass both embryonic stem cells (ESCs) and induced pluripotent stem cells (iPSCs).

The present invention is technically characterized by the utilization of neural precursor cells derived from pluripotent stem cells as the initial starting cells (source cells). Neural precursor cells offer greater ease of handling compared to pluripotent stem cells, and their predisposition to differentiate into neural cells expedites the process of inducing astrocytes. Further, neural precursor cells possess the advantage of being indefinitely expandable through subculture akin to general stem cells. As in the present invention, using neural precursor cells as starting cells provides the added benefit of enabling the large-scale production of astrocytes while minimizing the risk of contamination with other tissue cells (such as endodermal and mesodermal cells) following the differentiation into astrocytes.

As used herein, “neural precursor cells (or neural progenitor cells, hereinafter referred to as NPCs)” refers to cells in the earlier stages of development before neural cells have reached their final morphology and functionality. NPCs can differentiate from pluripotent stem cells. During this process, the expression of essential pluripotent transcription factors like OCT4 and NANOG decreases, and the distinctive feature of multiple cells growing in a colony diminishes. NPCs gradually adopt a small bipolar morphology with a columnar shape. Unlike pluripotent stem cells, which have the potential to differentiate into endoderm, mesoderm, and ectoderm cells, NPCs possess the capacity to differentiate exclusively into cells of the neuroectodermal lineage, including neurons, astrocytes, and oligodendrocytes. The NPC as per the invention may be a cell that co-expresses SOX1, SOX2, NESTIN, and PLZF. In particular, the NPC may be a cell that expresses SOX9 in conjunction with these factors. These genes are distinctively expressed in NPCs, and their expression thereof may signify that the resulting cells are NPCs.

In addition, in the present invention, the NPC may refer to an NPC into which no factors other than NFIB have been artificially introduced from external sources.

In the present invention, the gene encoding the NFIB protein may be introduced into NPCs using a vector. The term ‘vector’ refers to a genetic construct comprising external DNA inserted into a genome that encodes a polypeptide. This vector encompasses various types, including a DNA vector, plasmid vector, cosmid vector, bacteriophage vector, yeast vector, or viral vector, among others, without being restricted to these specific examples.

For example, the vector may be a viral vector or a plasmid vector. As an example of the viral vector, adenovirus, retrovirus, adeno-associated virus, herpes simplex virus, SV40, polyomavirus, papillomavirus, picornavirus, vacciniavirus, helper dependent adenovirus, or similar agents may serve as carrier for expressing NFIB. These viruses may also be recombinant viruses.

In an exemplary embodiment, the vector may incorporate a system that enables the control of NFIB expression, which can be introduced into the vector after in vivo introduction. For example, if a lentiviral vector is employed, NFIB expression may be modulated using an on-off regulator like doxycycline but is not limited thereto.

In the examples of the present invention, the gene was introduced into NPCs using a lentiviral vector as the viral vector for expressing NFIB, but vectors suitable for the present invention are not limited thereto.

In the present invention, a suitable expression vector includes a signal sequence or leader sequence for membrane targeting or secretion in addition to expression regulatory elements, such as a promoter, an operator, an initiation codon, a termination codon, a polyadenylation signal, and an enhancer, and may be prepared variously according to the purpose. The promoter of the vector may be constitutive or inducible. Further, the expression vector includes a selectable marker for selecting a host cell containing a vector and includes a replication origin in the case of being a replicable expression vector.

In one specific exemplary embodiment, when the NFIB gene is expressed through the viral vector or plasmid vector, an additional step allowing for the adjustment of NFIB gene expression may be included.

The NFIB gene to be introduced into the vector may be a nucleic acid sequence encoding an NFIB protein or an analog having characteristics functionally equivalent thereto. The nucleic acid may be DNA or RNA. In the present specification, an analog with functionally equivalent characteristics is defined as having features that are functionally equivalent to the NFIB. Specifically, it may be a peptide in which certain amino acid residues of the polypeptides or peptides exhibit a sequence homology (i.e., identity) of at least 80% or more, preferably 90% or more, with the amino acid sequence of NFIB or a fragment thereof, and may involve substitutions, deletion, or additions of amino acids. The substitution or deletion of the amino acid residues is preferably carried out in regions not directly involved in the biological activity of the NFIB polypeptide. The addition of the amino acid residues may involve appending several amino acids at both ends or within the sequence of the NFIB polypeptide. In addition, the analog with functionally equivalent characteristics, as described in the present invention, may encompass polypeptide derivatives in which the chemical structure of the polypeptide undergoes partial modification while preserving the fundamental framework of the NFIB polypeptide and its physiological activity. For example, the analog with functionally equivalent characteristics in this invention may incorporate structural adjustments aimed at modifying the stability, storage, volatility, or solubility of the NFIB polypeptide, among other features.

Furthermore, in the present invention, a publicly known transduction or transfection method may be used as a mean to introduce a vector expressing NFIB into a stem cell. Examples of such methods include centrifugation, microinjection, calcium phosphate precipitation, electroporation, liposome-mediated transfection, DEAE-dextran treatment, gene bombardment, and others. However, these examples are not exhaustive, and other methods may also be utilized.

In the present invention, the differentiation into neural cells may be achieved by methods typically performed in the art, such as a serum-free medium: treatment with morphogens such as FGFs, Wnt, and retinoic acid (RA), and the like, but the method is not limited thereto.

As used herein, the term “differentiation” refers to a process in which cells undergo specialization in structure or function as they divide, proliferate, and grow. This means that the morphology or function of cells, tissues, and other biological components within an organism change to fulfill their designated roles. In general, differentiation is a process through which a relatively simple system separates into two or more qualitatively distinct subsystems. For example, this can be observed in the development of qualitative differences between initially uniform parts of a biological system, such as the distinction between the head and body in the early stages of embryonic development or the differentiation of various cell types like muscle cells and neurons. This transformation results in the creation of qualitatively distinct regions or subsystems.

In the present invention, the differentiation into astrocytes can be achieved within a period as short as 2 weeks, or at most 3 weeks, for instance. According to the present invention, to achieve a high-yield, rapid differentiation into astrocytes, neural precursor cells were utilized as the starting cells, and NFIB alone served as the introduced factor. The present inventors not only discovered that neural precursor cells inherently express SOX9 but also observed that the introduction of NFIB upregulates the expression of endogenous SOX9. This confirmation underscores that the introduction of NFIB alone can rapidly and efficiently induce differentiation into astrocytes.

Further, the present invention provides a method for inducing differentiation from NPCs into astrocytes. The method involves introducing an NFIB protein or a gene encoding the same into the NPCs.

The differentiation process may also encompass the step of differentiating hPSCs into NPCs before introducing an NFIB protein or a gene encoding the same into the NPCs.

For example, the differentiation of hPSCs into NPCs may entail:
(a) culturing hPSCs for 8 days in a medium containing two SMAD inhibitors to induce the differentiation the hPSCs into NPCs; and
(b) isolating NPC-shaped cells, such as structures known as neural ‘rosettes’ from the cultured cells on day 8. Subsequently, the NPC-shaped cells are cultured at a high density in an NPC medium containing bFGF, but the method is not limited thereto.

Under the culture conditions described above, when NPCs reach their maximum density in an incubator, subculturing of NPCs can be achieved through dissociation using accutase. In such case, the subculture is characterized by a relatively short duration, for example, not exceeding 5 passages.

In the present invention, it is essential to utilize NPCs within 5 passages for optimal astrocyte differentiation yield. In addition, it is preferable to employ NPCs demonstrating a purity of 80% or higher, as indicated by SOX1 marker expression.

The previously mentioned information can be directly applied to hPSCs, NFIBs, NPCs, astrocytes, and similar elements in the method for inducing differentiation.

The method for inducing differentiation may involve the introduction of a gene encoding an NFIB protein into NPCs using a vector. Thereafter, it may encompass a step for culturing and differentiating the NFIB-introduced NPCs.

Specifically, the method for inducing differentiation may include:
(i) culturing NPCs in a basal medium containing bFGF, and then introducing NFIB;
(ii) culturing the NFIB-introduced NPCs in a basal medium containing CNTF and BMP4 for 1 to 3 days; and
(iii) culturing the NPCs cultured in (ii) in a medium for astrocyte differentiation for 9 to 18 days.

In Step (i), NPCs may be cultured in a basal medium containing bFGF for 15 to 24 hours, and then NFIB may be introduced. To introduce NFIB into NPCs, a method involving the introduction of a gene encoding an NFIB protein into NPCs through a vector may be used.

In Step (ii), an additional procedure may involve treating an on-off regulator for NFIB expression based on the type of vector used during the introduction of NFIB. Doxycycline can be used as the on-off regulator for NFIB expression, but it is not limited thereto.

In Step (iii), for the medium used in astrocyte differentiation during the differentiation of NPCs into astrocytes, any commercially available medium can be used, provided that it is designated for astrocyte differentiation.

In this case, in Steps (i) and (ii), a DMEM/F12 may be used as the basal medium, and Step (i) may be performed in a culture medium in which 1×N2 supplement, 1×B27 supplement and 10 to 30 ng/ml basic fibroblast growth factor (bFGF) are included in the DMEM/F12 basal medium, but the type of basal medium is not limited thereto. As the culture medium, typically used media for differentiation can be used without limitation.

In one exemplary embodiment, the basal medium may be a medium to which an epidermal growth factor (EGF) is not added.

In Steps (i) and (ii), the total number of culture days is preferably within 3 weeks, and may be, for example, within 2 weeks.

In the method for inducing differentiation according to the present invention, hPSC-derived NPCs (hereinafter referred to as hPSC-NPCs) serve as the initial cells. Approximately 75% or more of these cells may be differentiated into astrocytes within 3 weeks, particularly within 2 weeks, solely through the introduction of NFIB alone. Notably, in research in the related art, the shortest duration reported for differentiating from pluripotent stem cells into astrocytes is 4 weeks, with the longest duration extending to 1 year or more. The present invention offers a significant advantage over related research in the related art by shortening this differentiation period to 3 weeks or less.

Given that achieving high-purity and highly efficient differentiation is an essential element not only for basic research on cell development, differentiation, and functionality, but also for the future application of cell transplantation in therapeutic agent development, the present invention is deemed to possess significant technical merit.

In the present invention, “astrocytes” differentiated from NPCs may be characterized by the expression of one or more marker genes, including aquaporin 4 (AQP4), glial fibrillary acidic protein (GFAP), ryanodine receptor 3 (RYR3), insulin like growth factor binding protein 7 (IGFBP7), S100 calcium-binding protein β (S100β), aldehyde dehydrogenase 1 family member L1 (ALDHIL1) and CD44.

In addition, marker genes like GFAP, ALDH1L1, CD44 and S100β serve as indicators for the identification of astrocyte fate. These marker genes are characterized by an increasing level of expression during the progression of differentiation into astrocytes. As a result, these marker genes can serve as indicators, confirming the successful differentiation of hPSC-NPCs into astrocytes using the method for inducing differentiation described in the present invention.

In the present invention, GFAP-positive cells may constitute 80% or more of all differentiated cells two weeks after the initiation of the differentiation process in the method for inducing differentiation.

In the following examples, an analysis of expression levels, conducted by confirming the expression of GFAP and S100β through immunochemical analysis (FIG. 4-FIG. 4D), revealed that the cells positive for GFAP and S100β accounted for 84.48% and 84.77%, respectively, after 2 weeks of differentiation.

The maturation of astrocytes is crucial for the differentiated astrocytes in the present invention to effectively carry out their primary function, such as aiding in maintenance of neuronal function. Furthermore, mature astrocytes play a vital role in properly executing the function of glutamate uptake to eliminate glutamate released by neurons.

In the present invention, the astrocytes may be characterized by their ability to absorb glutamate, serving as an indicator of their maturity. In particular, the observation that the ability to absorb a certain level of glutamate is achieved approximately a week earlier than with the existing technique (Non-Patent Document 2) suggests that the differentiation of astrocytes in the present invention occurs more rapidly (FIG. 7A).

As described above, the astrocytes differentiated according to the present invention exhibit characteristics most similar to those of astrocytes isolated from the human adult brain.

In the following examples, through the confirmation and analysis of gene expression patterns, changes in intracellular calcium ion concentration, and the degree of glutamate uptake in astrocytes isolated from adult brain tissue and astrocytes differentiated according to the present invention, it was established that the astrocytes differentiated in this invention exhibit characteristics akin to those of astrocytes isolated from the actual human brain. In particular, this makes the present invention the first report where a majority of the astrocytes generated by the present invention demonstrate the all-or-none reactivity pattern observed in actual adult brain tissue, indicating the induction of calcium ion flow by a physiological cue (FIG. 6F). This observation attests that the present invention has achieved a physiological maturity comparable to that of astrocytes present in actual adult brain tissue.

The benefits and features of the present invention, and the methods of achieving the benefits and features will become apparent with reference to experimental examples and preparation examples to be described below in detail. However, the present invention is not limited to the experimental examples and the preparation examples to be disclosed below and may be implemented in various other forms, and the present disclosure is provided for rendering the disclosure of the present invention complete and for fully informing those with ordinary skill in the art to which the present invention pertains of the scope of the present invention.

### Advantageous Effects

A method for differentiating neural precursor cells into astrocytes, as per the present invention, is characterized by the overexpression of a transcription factor named NFIB alone while using NPCs as starting cells. This results in the differentiation of NPCs into astrocytes that exhibit a maturity level comparable to cells isolated from the adult brain within 2 weeks. This process mimics molecular and functional changes similar to the actual differentiation process of astrocytes in vivo. Therefore, the present invention not only offers a technique for efficiently producing astrocytes in a short period but also provides an effective research platform for studying the human astrocyte development process.

### DESCRIPTION OF DRAWINGS

FIG. 1 illustrates the characteristics of NPCs used for differentiation through immunofluorescence staining. A of FIG. 1 shows the expression of SOX1 and PLZF, representative markers for NPCs: and B of FIG. 1 shows the expression of NESTIN and SOX2, which are additional NPC markers. The nuclei of all cells were stained with 4′,6-diamidino-2-phenylindole (DAPI) to quantify the percentage of cells positive for each marker relative to the total number of cells (scale bar: 50 μm). C of FIG. 1 is a graph confirming and displaying the percentage of SOX1-positive cells, a definitive marker for NPCs, to validate the purity of NPCs used for astrocyte differentiation.

FIG. 2A-FIG. 2G shows that the overexpression of NFIB alone in hPSC-NPCs is sufficient to induce the fate of astrocytes. FIG. 2A illustrates a schematic view for the generation of astrocytes in hPSC-NPCs (BC: BMP4 and CNTF); FIG. 2B displays fluorescence images of GFAP- and SOX1-positive cells on day 14 of differentiation induced by the overexpression of the indicated transcription factors or combinations thereof (scale bar: 20 μm); and Panels A and B of FIG. 2C show the quantification of positive cells for the indicated markers on day 14 of differentiation. In particular, the results of FIGS. 2B and 2D indicate that the number of undifferentiated neural precursor cells (SOX1-positive cells) remaining after 14 days is smaller when NFIB is introduced alone compared to when NFIB and SOX9 are simultaneously introduced. This observation, indicating that fewer NPCs remain after differentiation, demonstrates that the introduction of NFIB alone is more advantageous from the viewpoint of astrocyte differentiation. FIG. 2D illustrates quantitative RT-PCR analysis results for SOX9 expression in hPSCs and hPSC-NPCs; FIG. 2E illustrates the average number of SOX9-positive cells in hPSCs and hPSC-NPCs quantified by random microscope fields: FIG. 2F illustrates representative images of the results of immunofluorescence staining of SOX9-positive cells in hPSCs and hPSC-NPCs (scale bar: 20 μm); and FIG. 2G illustrates the results of quantitative RT-PCR analysis on SOX9 expression in neural precursor cells with and without NFIB overexpression (n.s.: not significant: * p<0.05; p<0.01; and *** p<0.001).

FIG. 3 illustrates the results of astrocyte differentiation by introducing NFIB into NPCs differentiated from another hPSC line (i.e., a human embryonic stem cell line, H1). A of FIG. 3 displays cells positive for GFAP and S100β, and B of FIG. 3 shows the results of immunofluorescence staining illustrating cells positive for CD44 on day 14 of differentiation. C of FIG. 3 presents the percentage of cells positive for each marker based on the results of A and B of FIG. 3.

FIG. 4A-FIG. 4D illustrates the cellular and molecular characteristics of NFIB-induced astrocytes. Panels A, B of FIG. 4A depict fluorescence images for GFAP, S100β, and CD44, respectively), on day 14 of differentiation (scale bar: 50 μm); Panel C of FIG. 4A illustrates the quantification of cells positive for the indicated marker on day 14 of differentiation; Panel D demonstrates cells expressing hALDH1L1 using an EGFP-reporter system: FIG. 4B illustrates the results of quantitative RT-PCR analysis for the marker genes of astrocytes (GFAP, ALDHL1, CD44 and IGFBP7), neurons (MAP2), an oligodendrocyte progenitor (PDGFRα) and NPC (SOX1); and FIG. 4C and FIG. 4D present the results of comparing transcriptomes among NFIB-induced astrocytes, primary human astrocytes, astrocytes derived from hPSCs using other differentiation techniques, and other neural cells potentially found in the nervous system, through RNA sequencing. Specifically, FIG. 4C illustrates the results of PCA analysis, with the dashed circle highlighting NFIB-induced astrocytes; and FIG. 4D illustrates a heatmap of normalized read-counts, comparing genes related to the identity of astrocytes as provided in the previous documents (References 2 and 3 and Non-Patent Document 3) in NFIB-induced astrocytes and astrocytes provided in the previous documents (References 2 and 3 and Non-Patent Document 3).

FIG. 5A-FIG. 5D illustrates the expression dynamics of astrocyte-specific genes during differentiation induced by NFIB. FIG. 5A illustrates the results of expression dynamics through RT-PCR analysis of genes representing astrocytes (GFAP, ALDH1L1, CD44 and IGFBP7) during the differentiation process: FIG. 5B illustrates a heatmap of normalized read-counts showing genes for neurons and astrocytes: FIG. 5C illustrates a heatmap of normalized read-counts showing the changes in the expression of genes specifically expressed in adult- or fetal-derived astrocytes; and FIG. 5D presents the results of comparing the expression dynamics of selected adult genes (ALDH1L1, IGFBP7 and AGXT2L1) and fetal astrocyte genes (TOP2A, TMSB15A, and HIST1H3B) with previously reported results of identical gene expression dynamics observed during the astrocyte differentiation process conducted in the 3D-human cortical spheroid form over 590 days (Reference 5). This result indicates that the pattern of gene increase and decrease over 14 days by NFIB closely mirrors the existing pattern observed over 590 days. This suggests that astrocyte differentiation by NFIB mimics the known developmental process of astrocytes effectively, while significantly shortening the time period.

FIG. 6A-FIG. 6H shows results demonstrating the functional maturation of astrocytes in their calcium response to physiological stimulants with increasing in vitro age (differentiation period). FIG. 6A illustrates the results of GSEA analysis between day 0 of differentiation and day 14 of differentiation, showing the enrichment of genes associated with calcium-mediated signaling as the differentiation progresses: FIG. 6B displays a heatmap illustrating changes in expression of genes involved in calcium-mediated signaling (GO: 0019722); FIG. 6C and FIG. 6D illustrate images of the calcium response to 30 μM ATP and 100 μM glutamate in differentiated astrocytes on day 7 and day 14 of differentiation, respectively (scale bar: 50 μm): FIG. 6E and FIG. 6F illustrate a segment of the fluorescence ratio (F/FO) of cells recorded from FIG. 6C and FIG. 6D. Each trace illustrates a change in intracellular calcium levels within a single cell; FIG. 6G illustrates the percentage of cells exhibiting a calcium response of 3 (F/FO) or more under 30 μM ATP treatment in differentiated astrocytes on day 7 of differentiation and day 14 of differentiation; and FIG. 6H illustrates the percentage of cells exhibiting a calcium response of 10 (F/FO) or more under 100 μM glutamate treatment on day 7 and day 14 of differentiation. In particular, the calcium change trace graph on day 14 of differentiation (day 14) in FIG. 6F reveals a substantial number of cells exhibiting an all-or-none pattern, drawing a single curve (or double curve in some cases) in response to glutamate stimulation. According to Reference 3, this phenomenon is observed only in astrocytes in human adult brain tissue. This result indicates that the calcium response, a representative physiological function of astrocytes, is molecularly and functionally matured to the level of astrocytes in the actual adult brain on day 14 of differentiation.

FIG. 7A-FIG. 7F illustrates functional maturation for glutamate uptake and synaptosomal engulfment (or phagocytosis). FIG. 7A illustrates that the analysis of glutamate uptake from astrocytes on day 7 and day 14 of differentiation revealed a substantial improvement in their ability to remove glutamate from the culture medium as differentiation progressed: FIG. 7B displays a heatmap of gene expression related to neurotransmitter clearance during the period of astrocyte differentiation by NFIB, highlighting SLC1A3, ARL6IP5 and GLUL as genes directly associated with glutamate uptake in astrocytes: FIG. 7C illustrates images of cells that engulfed pHrodo-red-conjugated synaptosomes on day 7 and day 14 of differentiation (scale bar: 50 μm): FIG. 7D illustrates the results indicating a significant increase in the ability to engulf pHrodo-red-conjugated synaptosomes as differentiation progressed; FIG. 7E shows that GSEA on day 0 vs. day 14 revealed a clear enrichment of genes related to phagocytosis (GO: 0006909). FIG. 7F displays a heatmap of gene expression related to phagocytosis (GO:0006909) during the period of differentiation by NFIB, illustrating the progressive upregulation of most genes. In particular, a red box with an asterisk indicates several genes (MERTK and C3) known to play an important role in the removal of synapses. Data was presented as average+s.e.m (n=6 (FIG. 7A), n=5 (FIG. 7D)) (* p<0.05, ** p<0.01).

FIG. 8A-FIG. 8F demonstrates that the differentiation method, utilizing the overexpression of NFIB, can serve as a platform for discovering and studying crucial cell signaling mechanisms during the human astrocyte development process. FIG. 8A illustrates clustering and scaled expression patterns for differentially expressed genes (DEGs) during the differentiation process using NFIB. Notably, the gene groups represented by red boxes (Groups 6, 11, and 14) exhibit expression dynamics similar to GFAP and ALDH1L1. FIG. 8B shows the gene ontology (GO) analysis for the genes of Groups 6, 11 and 14, demonstrating an enrichment for transmembrane receptor protein kinase signaling and MAPK signaling pathways; FIG. 8C illustrates a schematic view of an experimental design involving the treatment of an inhibitor (U0126) targeting MEK1/2, a key enzyme in the MAPK signaling mechanism during astrocyte differentiation. FIG. 8D consists of fluorescence images for GFAP and SOX1 following differentiation based on the experimental design outlined in FIG. 8C, revealing a robust suppression of astrocyte differentiation through pharmacological inhibition of the MAPK signaling pathway (scale bar: 50 μm). FIG. 8E and FIG. 8F quantify the percentage of cells positive for each marker on day 14 of differentiation. These results demonstrate that astrocyte differentiation is highly sensitive to the inhibition of the MAPK signaling mechanism, establishing the MAPK signaling mechanism as a crucial cell signaling mechanism required for astrocyte differentiation. Consequently, the present data supports the notion that the astrocyte differentiation technique involving the overexpression of NFIB can serve as a valuable platform for molecular studies of the astrocyte developmental process. Data is presented as the mean #s.e.m. (n=3) (* p<0.05, ** p<0.01, *** p<0.001).

### MODES OF THE INVENTION

Hereinafter, the present invention will be described in detail with reference to the following examples. However, these examples are provided solely for the purpose of illustrating the present invention, and the scope of the invention is not limited by these examples.

### Example 1. Production of hPSC-NPCs and Differentiation into Astrocytes

1-1. hPSC-NPC Generation

An hiPSC cell line (NL1) and a human embryonic stem cell line (H1; WiCell, Madison, WI, USA) obtained from WiCell (USA) and were used for astrocyte differentiation. These hPSCs were cultured on a Matrigel-coated 6-well plate in the StemMACS™ iPS-Brew XF medium and treated with a rho-associated kinase (ROCK) inhibitor Y27632 (10 μM) to prevent dissociation-induced cell death. A slightly modified dual-SMAD inhibition strategy was used for differentiation into NPCs (Reference 1). Briefly, hPSC colonies were dissociated into single cells, and 1×10⁴ cells/cm² of the cells were plated on Matrigel-coated plates with StemMACS™ iPS-Brew XF medium containing 10 μM Y27632. On day 1 of differentiation into NPCs, cells were transferred to StemMACS™ iPS-Brew XF medium including 250 nM LDN193189 (Stemcell Technologies, Vancouver, BC, Canada) for suppressing bone morphogenetic protein (BMP) signals and 10 μM SB431542 (Millipore Sigma) for suppressing transforming growth factor-beta (TGF-β) signals and cultured for 8 days. On day 8 of differentiation, cells were dissociated with accutase (Thermo Fisher Scientific), resuspended in an NPC medium (DMEM/F12 medium supplemented with 1×N2 and 1×B27, all purchased from Thermo Fisher Scientific) containing 20 ng/ml bFGF (Pepro Tech, Rocky Hill, NJ, USA), and plated at a density of 3.5×10⁵ cells/cm² to prevent spontaneous differentiation. The hPSC-NPCs produced in this manner could be subcultured using accutase at a consistent density. These cells were suitable for differentiation into astrocytes when immunochemical analysis revealed that 80% or more of the cells were positive for SOX1, a definitive marker for NPCs. The cells with passage number less than 5 are used for the differentiation into astrocytes. Media were replenished every other day.

### 1-2. Production of Lentiviral System

In the following experimental example, plasmids for NFIB, SOX9 and hALDH1L1-EGFP expression were constructed to determine the optimal combination of transcription factors for hPSC-NPCs.

To clone a plasmid for NFIB expression, a T7-VEE-GFP plasmid (#58977, Addgene) was cleaved with a restriction enzyme Xbal (New England Biolabs, Ipswich, Mass., USA) to obtain an IRES-PuroR fragment. After cleaving TetO-FUW-NfiB (#64900, Addgene) with the same restriction enzyme, the IRES-PuroR fragment was inserted using a traditional ligation method, resulting in TetO-FUW-NfiB-IRES-Puro®.

To clone a plasmid for SOX9 expression, TetO-FUW-DLX2-IRES-hygro® (#97330, Addgene) was cleaved with restriction enzymes (EcoRI and BamHI, both from New England Biolabs) to remove the open reading frame (ORF) of DLX2; and then the ORF of SOX9 cloned from hPSCs derived from NPCs was inserted using a traditional ligation method, resulting in a plasmid called TetO-FUW-SOX9-IRES-hygro®. A lentiviral vector including a reverse tetracycline controlled trans-activator (rtTA) was purchased from Addgene (#20342).

To clone a plasmid for hALDH1L1-EGFP, a human ALDH1L1 promoter region was cloned from human genomic DNA. The plasmid of pcDH-pigGFAP-EGFP-EF1a-Puro® was cleaved with restriction enzymes, Spel and BamHI (New England Biolabs); and then a cloned human ALDH1L1 promoter region was inserted using a traditional ligation method. 293FT cells (Thermo Fisher Scientific) were transfected with a plasmid containing NFIB, SOX9, hALDH1L1-EGFP or rtTA, packaging vectors pMDLg/pRRE, pRSV-Rev and envelope PMD2.G (#12251, #1225, and #12259, respectively, all purchased from Addgene). A virus-containing culture medium was harvested 72 hours after transfection and concentrated using a Lenti-X concentrator (Takara, Nojihigashi, Kusatsu, Japan). After titration, the concentrated suspension of virus particles was aliquoted for further use.

1-3. Differentiation from hPSC-NPCs into Astrocytes

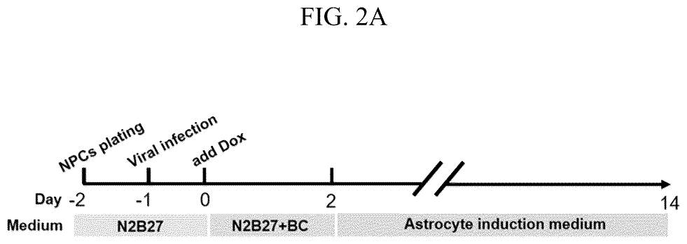

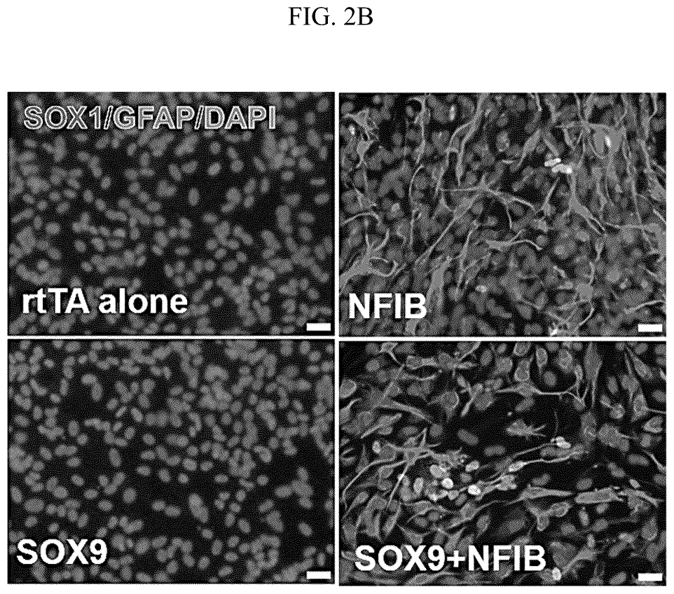

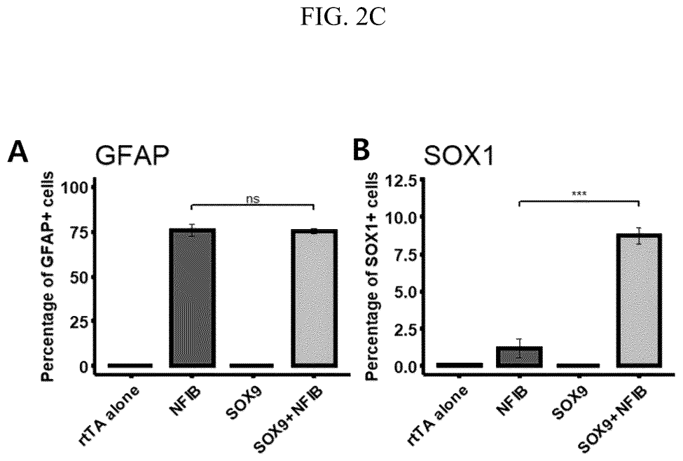

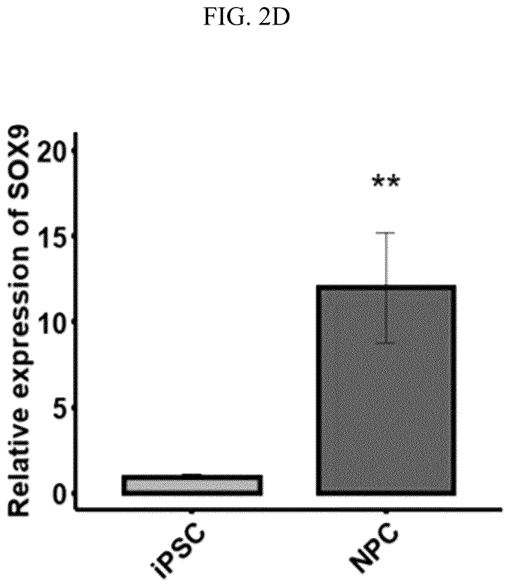

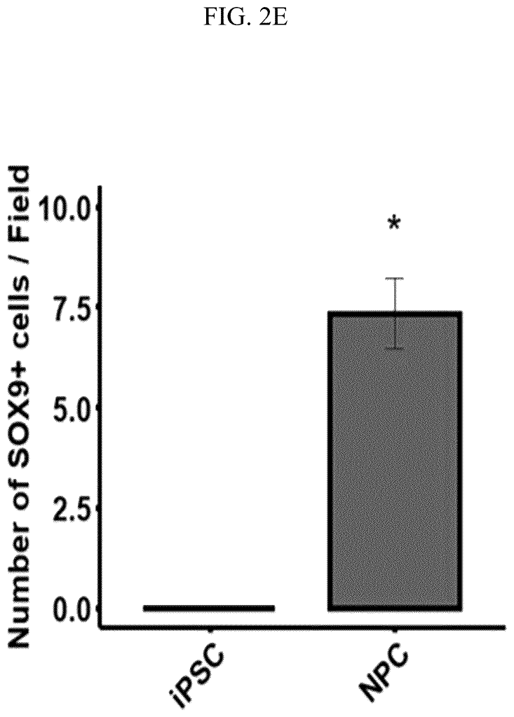

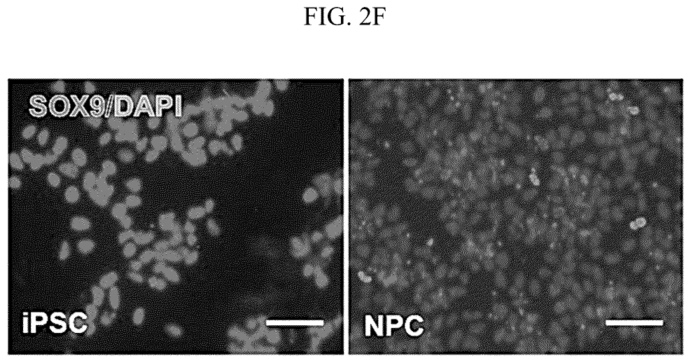

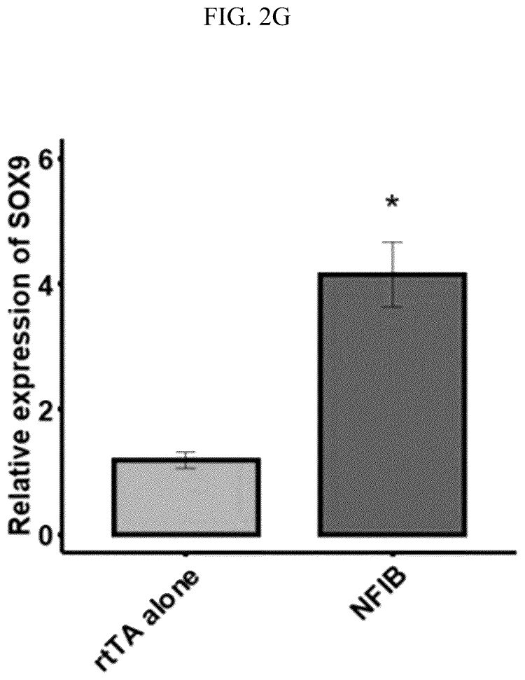

For viral infection, hPSC-NPCs with a passage number less than 5 were seeded at a density of 3×10⁴ cells/cm² on a Matrigel-coated 6-well plate and cultured for a day to ensure uniform and complete attachment of all cells to the bottom of the plate. Subsequently, cells were infected with a virus (inoculated virus dose/cell counts, multiplicity of infection (MOI): 1.0) and 1 μg/ml polybrene (Millipore Sigma). Plates with attached cells were centrifuged at 1000 g at room temperature (RT) for 1 hour in order to improve transduction efficiency during virus infection. After 18 hours of culture, the virus-containing medium was replaced with a fresh NPC medium containing 10 ng/ml ciliary neurotrophic factor (CNTF), 10 ng/ml BMP4 (Protech), and 2.5 μg/ml doxycycline (Millipore Sigma) to induce NFIB expression. In this case, the day when doxycycline was added was designated as day 0 of differentiation (FIG. 2A). On day 2 of differentiation, the NPC medium was replaced with a commercially available astrocyte medium (AM; ScienCell, Carlsbad, CA, USA), and this medium was used up to day 14 of differentiation. Positive selection for virus-infected cells was carried out from days 1 to 5 using 1.25 μg/ml puromycin and/or 200 μg/ml hygromycin (Thermo Fisher Scientific).

### Experimental Example 1. Confirmation of Efficiency of Differentiating hPSC-NPCs into Astrocytes

### 1-1. Confirmation of Characteristics of NPCs Used in Astrocyte Differentiation

The fundamental characteristics of NPCs used for differentiation into astrocytes in the present invention were validated through immunofluorescence staining.

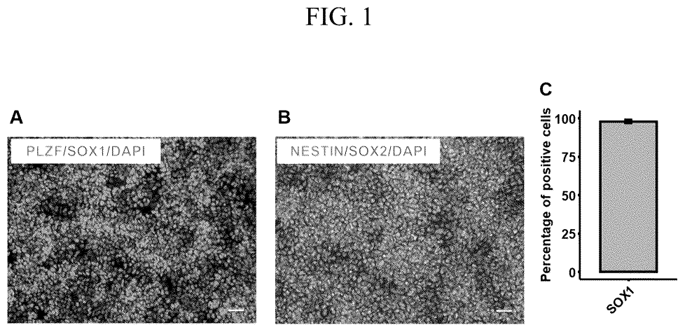

As depicted in FIG. 1, it was confirmed that SOX1 and PLZF, representative markers expressed in NPCs, were expressed (FIG. 1A), and NESTIN and SOX2 were also expressed (FIG. 1B). In addition, the number of SOX1-positive cells was quantified as a percentage of the total cell count (FIG. 1C).

### 1-2. Determination of the Optimal Combination of Transcription Factors

A recent study (Neuron. 2013 78(5):785, doi: 10.1016/j.neuron.2013.05.029) has demonstrated the rapid and efficient generation of desired neurons from stem cells through the overexpression of specific transcription factors. In conceptually similar studies, it was found that the overexpression of SOX9, NFIA, NFIB and the combination of these factors resulted in the induction of astrocytes from human fibroblasts (Reference 6) or hPSCs (Non-Patent Document 2) within just a few weeks. Another recent study (Non-Patent Document 3) induced astrocytes over about 8 weeks by introducing NFIA into hPSC-derived LTNSCs and analyzed the molecular mechanisms of astrogliogenesis using this system. These findings clearly demonstrate that NFIB (alone or in combination with SOX9) and/or NFIA are transcription factors capable of differentiating hPSCs and LTNSCs into astrocytes, respectively.

However, in contrast to the established role of NFIB and NFIA as transcription factors involved in the differentiation of hPSCs and LTNSCs into astrocytes, recent studies have reported that elevated expression of NFIB in NPCs or neural stem cells promotes differentiation into oligodendrocytes rather than astrocytes (References 7 and 8). Nevertheless, it was determined that hPSC-NPCs offer an advantage as starting cells for producing neural cells compared to hPSCs, given that, unlike hPSCs, hPSC-NPCs are less likely to differentiate into endo/mesodermal cells due to their predetermined fate as neural cells. Therefore, the present inventors needed to first confirm whether the overexpression of NFIB in hPSC-NPCs can induce astrocyte differentiation or whether additional factors are required. Simultaneously, the present inventors aimed to identify an optimal transcription factor or combination capable of more rapidly and efficiently producing astrocytes than existing methods, utilizing hPSC-NPCs as starting cells.

Firstly, in the case of NPCs differentiated from hiPSC cells (NL1), SOX9, NFIB, or a combination thereof, which have not been used in hPSC-NPCs in the related art, were introduced using a doxycycline-inducible lentiviral system (FIG. 2A). As a result, it was confirmed that the overexpression of NFIB alone and the overexpression of SOX9 and NFIB combination can efficiently induce cells expressing GFAP, an astrocyte marker, in NPCs within 2 weeks (75.89% and 75.40% in total cells, respectively) (FIG. 2B, and Panel A of FIG. 2C). However, interestingly, it was found that, compared to the experimental group introduced with NFIB alone, more undifferentiated NPCs (SOX1-positive cells) remained in the experimental group introduced with the combination of SOX9/NFIB (FIG. 2B and Panel B of FIG. 2C), which had previously shown the most effective differentiation into astrocytes when introduced into hPSCs, after 2 weeks of differentiation.

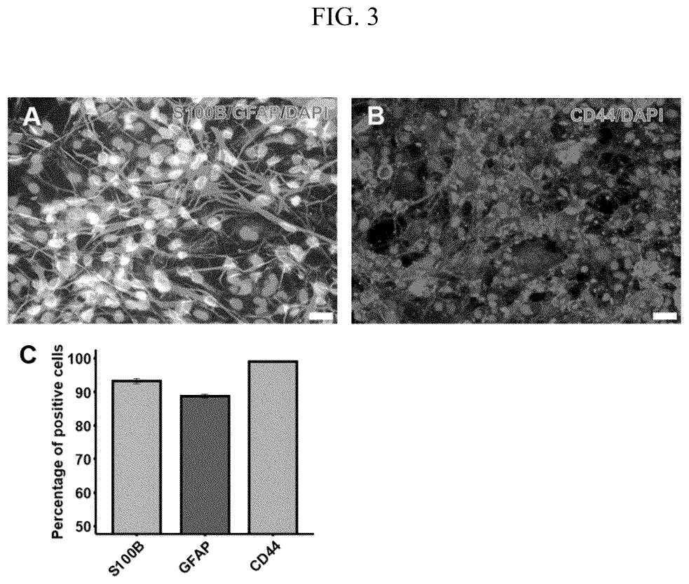

Subsequently, NPCs differentiated from human embryonic stem cells (H1) were also differentiated into astrocytes by introducing NFIB in the same manner as hiPSC-derived NPCs. As illustrated in FIG. 3, similar to the hiPSC cells, the cells were positive for all markers (GFAP, S100β and CD44) (FIGS. 3A and 3B) and showed a yield of about 90% for the markers (FIG. 3C).

According to Reference 9, SOX9 is recognized as a factor essential for maintaining the self-renewal ability of neural stem cells. This forms the basis for inferring that the overexpression of SOX9 in NPCs not only directs cells toward glial fate but also preserves the undifferentiated state of the cells, leading to an increased presence of SOX1-positive cells. However, it has been established, as demonstrated in Reference 10, that SOX9 is necessary for astrocyte differentiation. Therefore, the present inventors hypothesized the existence of a basal expression level of SOX9 in hPSC-NPCs, suggesting that the overexpression of NFIB might play a role in inducing astrocyte differentiation through modulating SOX9 expression. Indeed, as depicted in FIG. 2D, FIG. 2E and FIG. 2F, it was confirmed that the basal expression of SOX9 was higher in hPSC-NPCs than in hPSCs. In addition, it was verified that the overexpression of NFIB in hPSC-NPCs further elevated the basal expression level of existing SOX9 (FIG. 2G). These findings suggest that hPSC-NPCs inherently express SOX9, and the introduction of NFIB enhances the endogenous expression level of SOX9. This heightened expression level, in addition to the originally basal expression level, is sufficient to induce the differentiation of hPSC-NPCs into astrocytes without the side effect of retaining the undifferentiated state. Therefore, the innovative concept of differentiating astrocytes through the sole overexpression of NFIB in hPSC-NPCs was successfully established.

1-3. Characterization of Astrocytes Derived from hPSC-NPCs

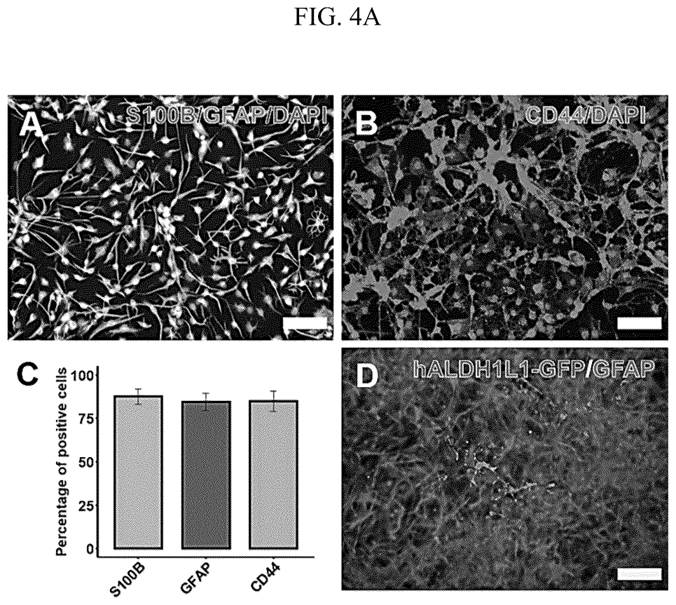

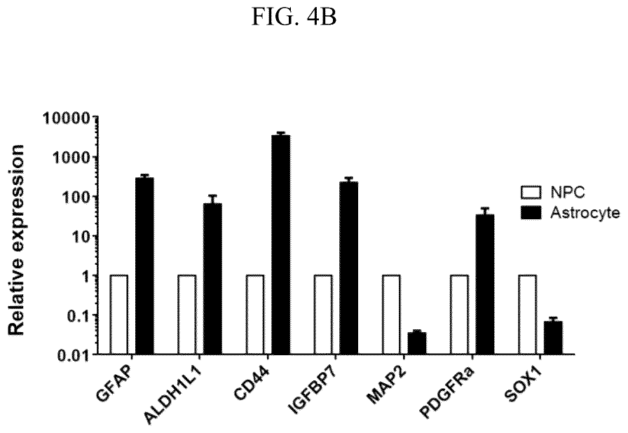

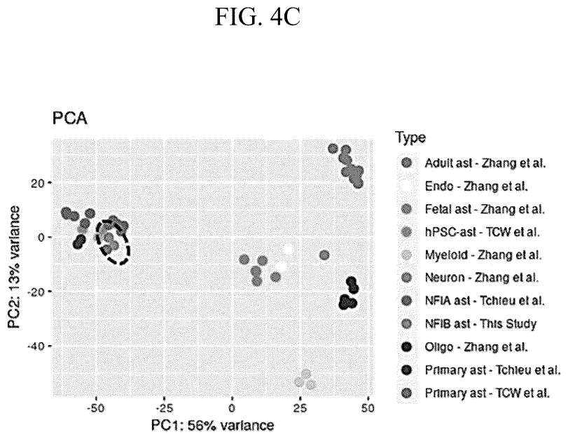

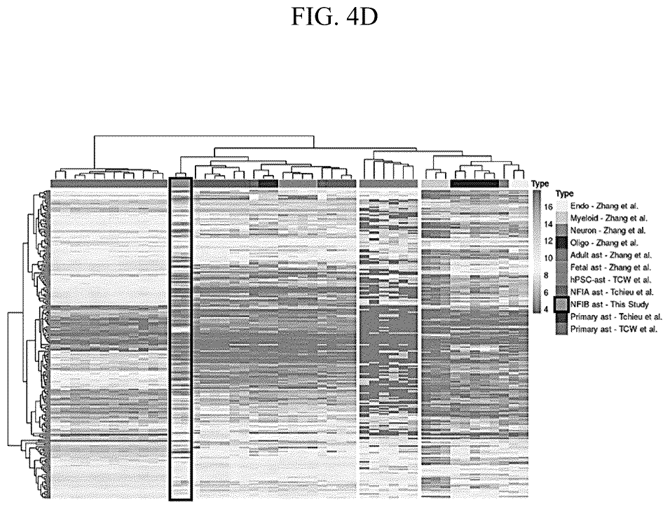

The GFAP-positive cells induced by introducing NFIB exhibited a stellate shape with a slender soma and multiple processes, similar to the morphology of the quiescent human primary astrocytes (Panel A of FIG. 4A).

To verify that the differentiated GFAP cells were authentic astrocytes, immunofluorescence staining was performed on various astrocyte markers. For immunofluorescent staining, cells cultured on the glass coverslips were fixed in 4% paraformaldehyde (PFA) for 15 minutes at room temperature (RT) and washed with phosphate-buffered saline (PBS). After permeabilization with 0.05% Triton X-100 in PBS for 10 minutes, the cell samples on the coverslips were blocked using a 2% bovine serum albumin solution or 5% donkey serum solution (both diluted with PBS) for at least 1 hour and then incubated with primary antibodies (see below) at 4° C. overnight. After thoroughly washing with PBS, the cultured samples were incubated with appropriate secondary antibodies conjugated with a fluorescent dye (Alexa Fluor® 488 or 594, Thermo Fisher Scientific) for 30 minutes at room temperature (RT). Samples for fluorescence microscope observation were completed by mounting the coverslips on glass slides using a mounting solution (Vector Laboratory, Burlingame, CA, USA). Cell images for analysis were obtained using a fluorescence microscope (IX71) equipped with a digital camera (DP71) (both from Olympus, Shinjuku, Tokyo, Japan). Primary antibodies used in this study were as follows: SOX1 (Goat, 1:200; R&D Systems, Minneapolis, MN, USA), SOX9 (Rabbit, 1:200; Abcam, Cambridge, United Kingdom), GFAP (Rabbit, 1:1000; Dako, Santa Clara, CA, USA), S100β (Mouse, 1:1000; Millipore Sigma), CD44 (Rat, 1:100; Thermo Fisher Scientific), MAP2 (Mouse, 1:1000, Thermo Fisher Scientific) and 04 (Mouse, 1:400; R&D Systems).

The immunofluorescence staining revealed that 84.48%, 87.67%, and 84.77% of total cells were positive for GFAP, S100β, and CD44, respectively, reaffirming that the majority of cells acquired the astrocyte fate (Panels A to C of FIG. 4A). To observe the expression of ALDH1L1 used as a marker for mature astrocytes, an ALDH1L1 reporter lentivirus (hALDH1L1-EGFP, “h” stands for “human gene”), which exhibits green fluorescence (EGFP) when ALDH1L1 gene is expressed, was prepared. Upon introducing ALDH1L1 reporter lentivirus into differentiated cells, it was confirmed that a plurality of GFAP-positive cells was strongly green-fluorescent. These results demonstrated that ALDH1L1-expressing mature astrocytes appeared as early as 2 weeks after NFIB expression (Panel D of FIG. 4A).

To verify whether the present differentiation strategy selectively differentiated only astrocytes, a quantitative gene expression analysis was performed on various markers for different cells present in the nervous system, including neurons, oligodendrocytes, astrocytes, and NPCs. As expected, the upregulation of astrocyte marker genes such as GFAP, ALDH1L1, CD44, and IGFBP7 was evident (FIG. 4B). However, the expression of the markers for NPCs (SOX1-positive) and neurons (MAP2-positive) was significantly decreased (FIG. 4B). This indicates that the majority of cells were induced into astrocytes.

To further confirm the identity as astrocytes and to confirm the extent to which NFIB-induced astrocytes share molecular characteristics with human primary astrocytes and hPSC-induced astrocytes from other studies, transcriptome data was obtained from astrocytes by RNA sequencing (RNA-seq) at 2 weeks of differentiation and then compared with the astrocyte transcriptomes thereof reported and disclosed in various existing documents (Non-Patent Document 3 and References 2 and 3). For RNA-seq analysis, the total RNAs were first extracted using TRIzol (Thermo Fisher Scientific) from astrocytes differentiated for 2 weeks through NFIB introduction. Then, the total RNA was submitted to E-biogen Inc. (Seoul, Korea) to collect RNA-seq raw data. The raw FASTQ files were trimmed for adapters using BBDuk (Reference 4) and aligned with the ENSEMBL GRCh38 genome build by using STAR (2.7.2a). Matrices were generated from the aligned files and imported into the DESeq2 (1.27.12) for further analysis using a standard pipeline. The raw FASTQ files of hPSC-derived astrocytes and human primary astrocytes published in existing literature were obtained from the GEO database (accession numbers GSE73721, GSE97904, and GSE104232) and then subjected to a pre-treatment process with the same pipeline previously mentioned. Subsequently, a correlation plot was prepared using ggplot2 (v3.3.0), and a heatmap was drawn using pheatmap (v1.0.). Gene Ontology (GO) enrichment analysis was performed using Metascape, if necessary. Gene set enrichment analysis (GSEA) was conducted using GSEA v4.0.3 (Broad Institute, Cambridge, MA, USA).

To intuitively compare and confirm the similarity with the previously reported transcriptomes of astrocytes, principal component analysis (PCA) using the entire transcriptome was initially conducted. Consequently, the transcriptomes of astrocytes differentiated by introducing NFIB still clustered distinctly from those of primary astrocytes purified by HepaCAM-mediated isolation method from human adult or fetal brain tissue. However, they exhibited close clustering with human primary fetal astrocytes reported in other literatures and with hPSC-derived astrocytes differentiated by other methods (FIG. 4C, indicated by a black dotted ellipse). Furthermore, an analysis demonstrating the similarity of each cell group through hierarchical clustering was conducted by comparing the degrees of RNA expression for all gene groups associated with astrocytes, a frequently used transcriptome comparative analysis technique in the art. Consistent with the previous PCA analysis, it was demonstrated that astrocytes differentiated by introducing NFIB were closely positioned to human primary fetal astrocytes and hPSC-derived astrocytes differentiated by other methods (FIG. 4D). As expected, various cell types that could be isolated from nervous tissues, such as neurons, oligodendrocytes, cerebrovascular endothelial cells, and ependymocytes, exhibited a lower correlation with the NFIB-induced astrocytes. This underscores that the astrocytes induced by introducing NFIB are highly similar at the molecular level to astrocytes differentiated by methods well-known in the related art.

These results suggest that astrocytes differentiated by introducing NFIB in hPSC-NPCs exhibit molecular characteristics similar to astrocytes isolated from the brain tissue of an actual human adult and other previously reported hPSC-derived astrocytes.

1-4. Analysis of Molecular Developmental Process of hPSC-NPC-Derived Astrocytes

Based on existing literature and the evidence presented by the present invention, differentiation through the forced expression of transcription factors has been established as a rapid and robust method for generating astrocytes. However, it remains unclear whether this paradigm aligns with the actual astrocyte differentiation program in vivo. Numerous studies on neurodevelopmental diseases have indicated that abnormal astrocyte differentiation processes contribute to these diseases. Consequently, when generating astrocytes for the purpose of studying related diseases, it is crucial to execute the proposed differentiation method in accordance with the authentic differentiation program.

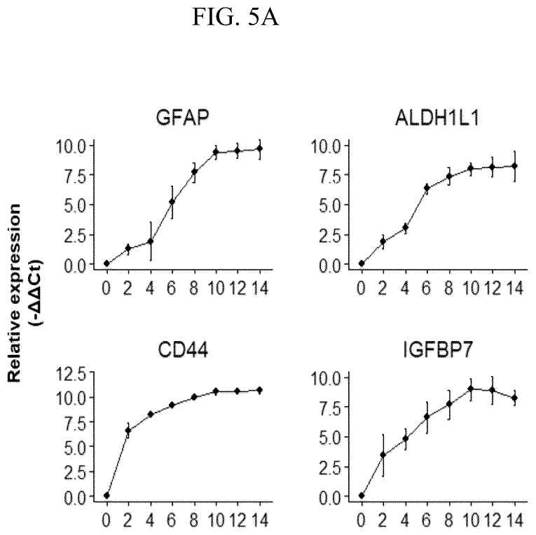

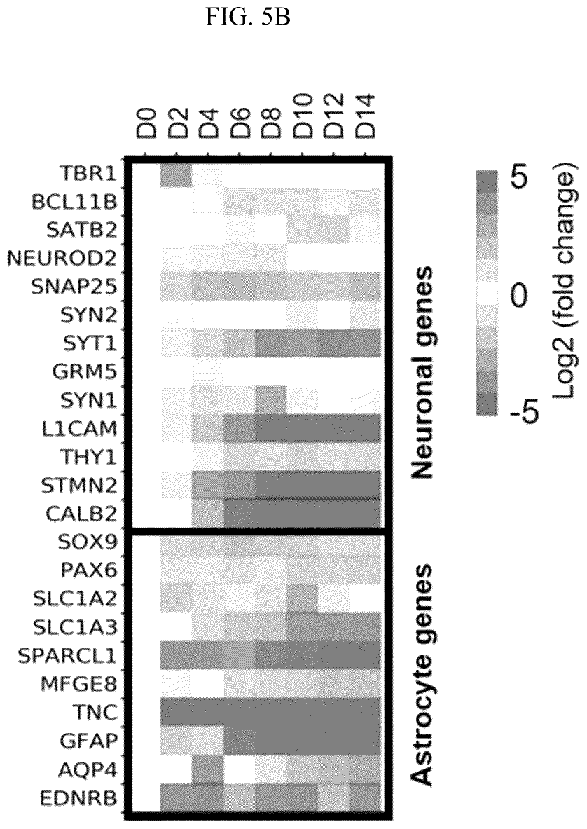

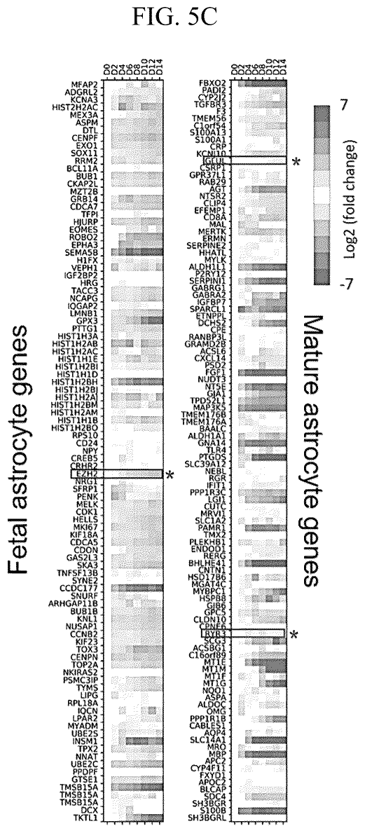

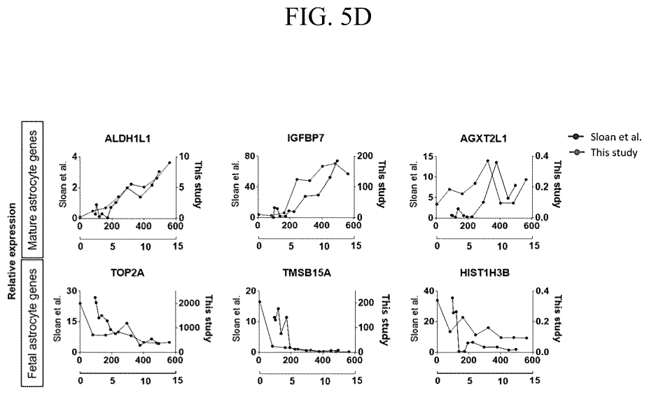

To verify whether the differentiation method proposed by the present invention aligns with the differentiation process of astrocytes that occurs in vivo, the expression dynamics of representative astrocyte marker genes was assessed during differentiation using quantitative RT-PCR. As illustrated in FIG. 5A, all astrocyte marker genes were gradually increased over the course of 2 weeks of differentiation.

In particular, the expression of CD44 instantaneously increased upon induction of NFIB, while GFAP and ALDH1L1 exhibited a somewhat delayed increase, which became more pronounced after 4 days. This phenomenon is attributed to the fact that the expression of CD44 precedes that of GFAP in the developing nervous system, as described in Reference 11. These expression dynamics provide an initial premise that cells differentiated by the present method follow a pattern similar to the actual developmental process. Expression of all the tested genes reached a maximum on day 10 and plateaued thereafter. This expression pattern suggests that the acquisition of astrocyte fate begins immediately after the overexpression of NFIB and completes at around day 10, after which functional maturation may occur.

To conduct a more in-depth analysis of the cell differentiation process at the molecular level, changes in transcriptomes were observed at various time points through RNA-seq data analysis over the 2-week period during astrocyte differentiation. Initially, when examining the expression of gene sets specific for neurons or astrocytes, it was observed that astrocyte-specific genes such as GFAP and aquaporin 4 (AQP4) were upregulated, while neuron-specific genes such as specific AT-rich sequence-binding protein 2 (SATB2), synapsin 1 (SYN1), stathmin-2 (STMN2), and L1 cell adhesion molecule (LICAM) were downregulated (FIG. 5B). This result indicates that the differentiation process induced by NFIB is characterized by biased genetic programming toward astrocytes rather than neurons.

The gene sets for analysis were expanded to include those specific to astrocytes: the top 50 genes specifically expressed in fetal astrocytes, and another top 50 genes specific to mature adult astrocytes were tested. Heatmaps of normalized read counts showed a clear trend, with most fetal astrocyte-specific genes gradually downregulated, while mature astrocyte genes were prominently upregulated (FIG. 5C). Interesting changes in specific gene expression were noticed: for example, the expression of the enhancer of zeste homolog 2 (EZH2), a gene known to a direct downstream target of NFIB, was downregulated. EZH2 is known to regulate the differentiation of the cortical neural progenitors and the expression of the astrocyte genes (Reference 12). Additionally, the upregulation of ryanodine receptor 3 (RYR3), a gene specific to mature astrocyte, was noteworthy, as it is one of the genes that distinguish human astrocytes from mouse astrocytes.

Reference 5 reported that astrocytes, differentiated for approximately 590 days through the formation of 3D cerebral cortical spheroids (organoid-like structures), closely mimicked the molecular and physiological characteristics of human astrocyte development. Based on the transcriptome change data spanning 560 days obtained from astrocytes differentiated through 3D cerebral cortical spheroids, as published in Reference 5, a direct comparison was attempted to assess the similarity of the changes in transcriptome dynamics between astrocytes differentiated through NFIB transduction and those differentiating in the 3D cerebral spheroids. For this purpose, the expression patterns of several genes were compared by adjusting and superimposing the changes within the same time frame (FIG. 5D). Despite the absolute time difference (14 days in the present invention vs. 590 days in Reference 5), the aligned expression patterns showed a surprisingly similar trend in the transcriptome dynamics, suggesting that the differentiation induced by the overexpression of NFIB in hPSC-NPCs occurs molecularly similarly to that observed in 3D cerebral cortical spheroids. Moreover, a comparison of the expression pattern demonstrated that the year-long molecular change patterns for astrocyte differentiation were remarkably compressed down to just 2 weeks by the forced differentiation through the NFIB overexpression (FIG. 5D). Furthermore, the gradual downregulation of DNA topoisomerase 2-α (TOP2A), a cell proliferation marker, indicated a persistent decrease in cell division during the process of differentiation into astrocytes. Numerous studies have already confirmed that this is a general phenomenon occurring in the astrocyte differentiation process. These lines of evidence collectively show that NFIB-induced differentiation is not the result of a simple drastic change in cytoplasm, but rather specifically and rapidly generates astrocytes from NPCs while following the normal developmental program of the original cell.

1-5. Confirmation of Functional Maturation of hPSC-NPC-Derived Astrocytes

### 1) Calcium Transient Assay

One of the physiological features of normal astrocytes is to retain the ability to change intracellular calcium concentration in response to extracellular stimuli. In particular, this function is critical for the astrocytes to modulate synaptic transmission and blood flow in the nervous system.

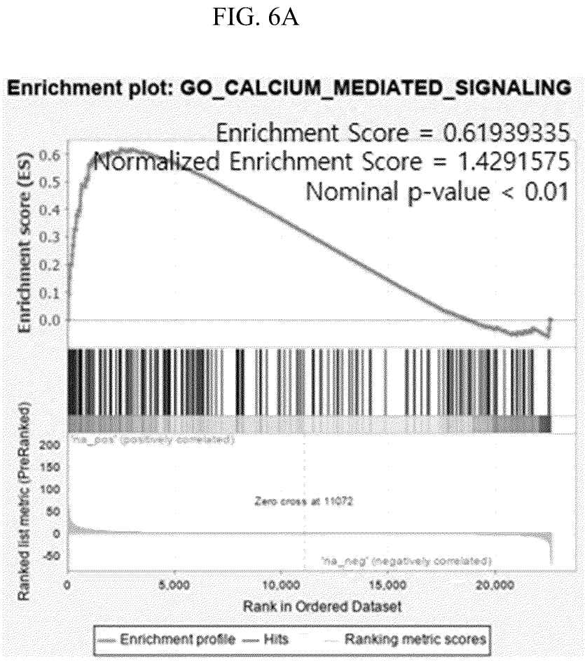

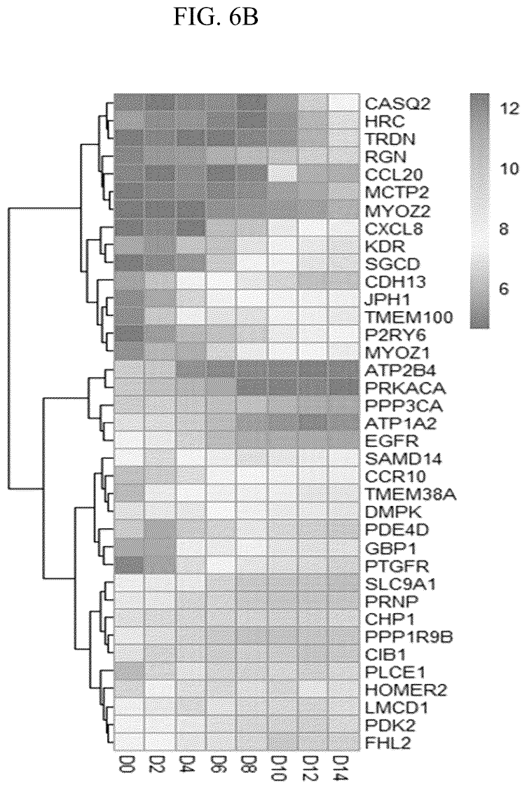

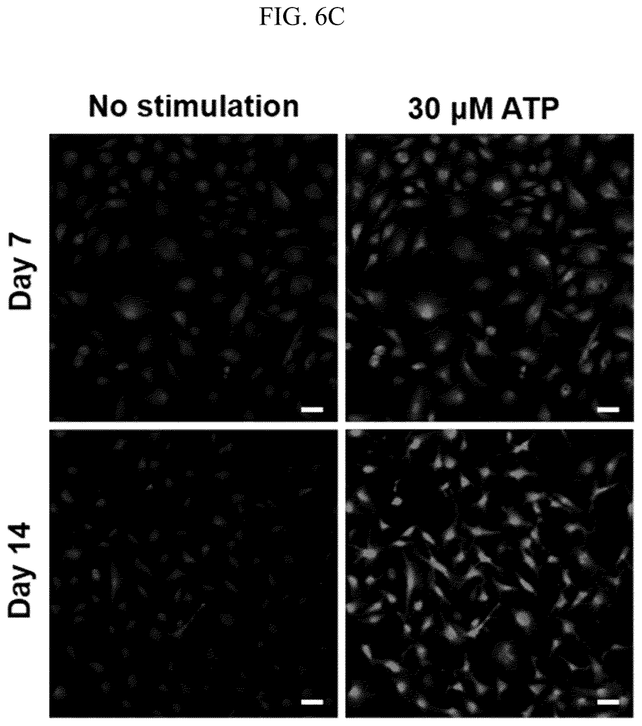

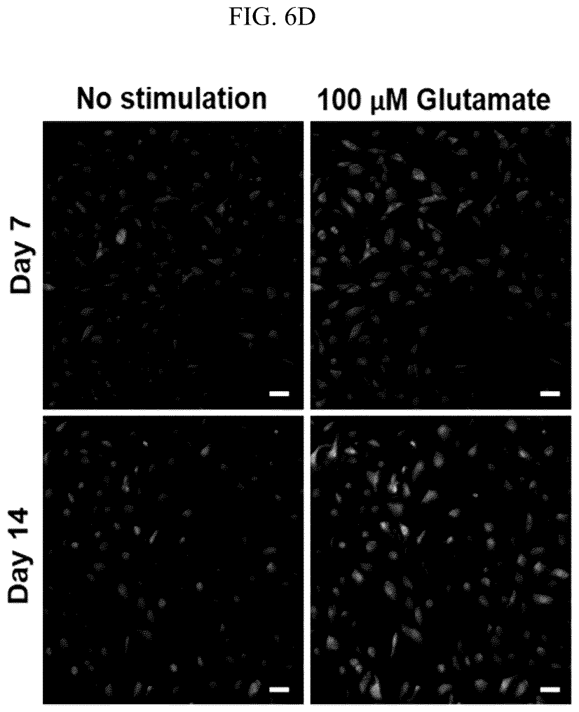

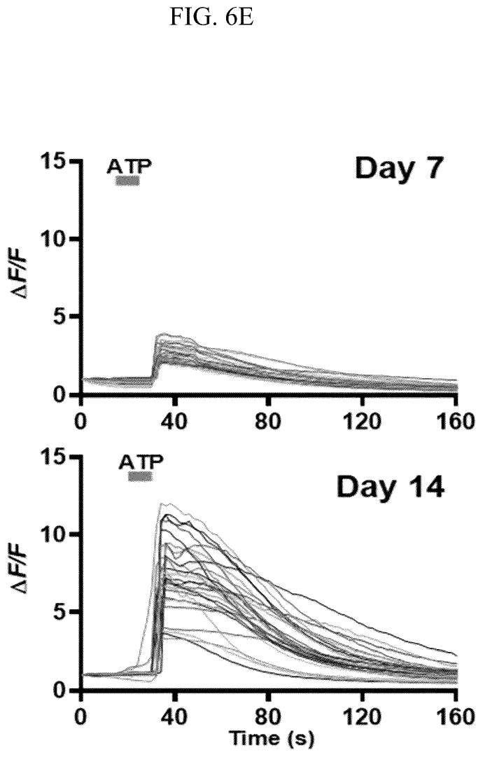

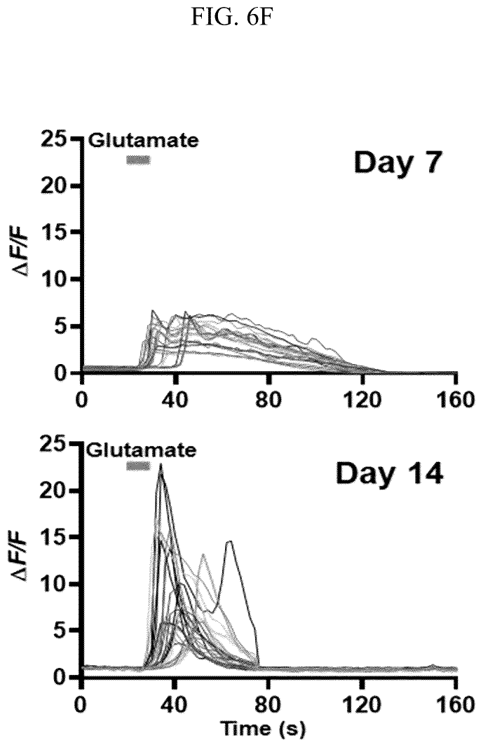

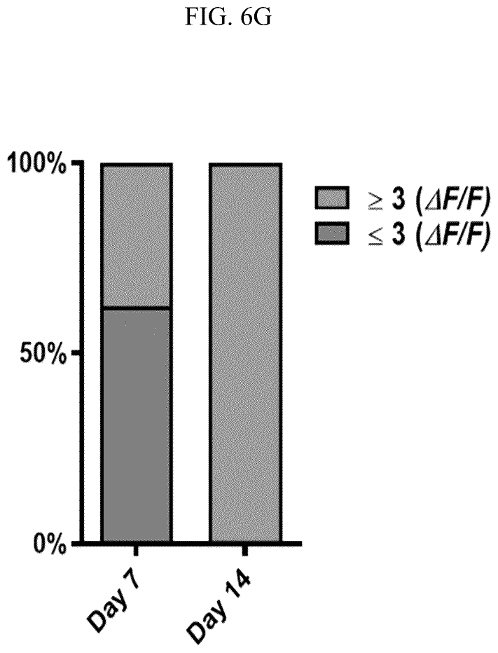

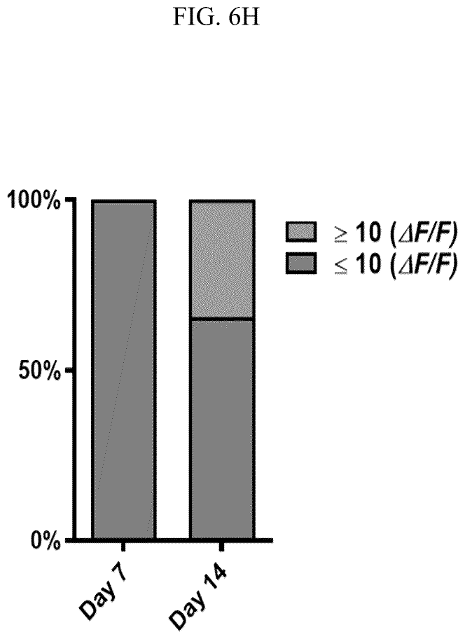

To confirm whether the cells acquire the ability to change intracellular calcium concentration, gene set enrichment analysis (GSEA) was employed to validate the expression pattern of genes related to this specific function. This analysis involved comparing the ranked gene signature of day 0 cells versus day 14 cells, with genes related to calcium-mediated signaling (GO:0019722). The analysis revealed a significant shift in the gene set associated with calcium-mediated signaling (FIG. 6A). Moreover, the heatmap illustrating the expression of these genes showed a gradual upregulation during differentiation (FIG. 6B). Based on these results, the response of NFIB-induced astrocytes to ATP and glutamate, specifically, the presence or absence of calcium increase in astrocytes in response to physiological stimuli, was examined. Calcium imaging was conducted on cells on days 7 and 14 using Fluo-4, a calcium indicator, and the intracellular calcium transients between two time points were compared. For calcium imaging, the differentiated astrocytes were prepared on 12 mm coverslips. After replacing the cell culture medium with Hank's Balanced Salt Solution (HBSS, without calcium or magnesium) (Thermo Fisher Scientific), differentiated astrocytes were incubated with 5 μM Fluo-4 AM (Thermo Fisher Scientific) at 37° C. for 30 minutes and then washed twice with the HBSS solution before calcium imaging. Thereafter, intracellular calcium levels were analyzed using the Fluo-4 AM fluorescence dye detection method, employing a confocal microscope (Fluoview FV1000, Olympus). For this purpose, intensity images of 535 nm wavelength were captured at 488 nm excitation wavelength at a rate of one frame per 2 seconds. Intracellular calcium levels were expressed as a fluorescence ratio (F/FO), calculated as changes in the fluorescence intensity (F) after treatments with 30 μM ATP or 100 μM glutamate, compared to the initial fluorescence intensity (FO) in the resting state.

As evident in the images in the left-column of FIG. 6C and FIG. 6D, the cells exhibited spontaneous calcium influx without external stimulation on both days 7 and 14 of differentiation. However, upon exposure to ATP, the intracellular calcium level was instantly elevated in almost all cells and sustained for 50 seconds or more, regardless of the differentiation period of sample cells (FIG. 6C and FIG. 6E). The amplitudes of calcium transients, expressed as a fluorescence ratio (F/FO), were less than 3 in the majority of the cells on day 7 of differentiation (84.3% among total cells, n=70); however, it increased to 3 or more in all the cells tested on day 14 of differentiation (n=80) (FIG. 6E and FIG. 6G). This result indicated that the responsiveness to ATP increased as the cells matured over time. Subsequently, the responsiveness of the cells to glutamate was tested. When exposed to glutamate, the majority of cells on day 7 of differentiation showed an immediate elevation of intracellular calcium concentration with asynchronous oscillations lasting less than 100 seconds, and the average amplitude was 4.92±0.32 (n=19) (FIG. 6F). In contrast, in the astrocytes on day 14 of differentiation, glutamate stimulation produced single (in most cases) or double intracellular calcium peaks with much greater amplitudes (9.63±1.07, n=26) (FIG. 6F) and one-third of all the cells tested (32.4%) showed amplitudes greater than 10 (FIG. 6H). These “all-or-none”-like responses to glutamate stimulation were reported only in human adult astrocytes in the related art but never in human fetal astrocytes before mid-gestation (Reference 3). Furthermore, this is a result that has not previously been reported in any hPSC-derived astrocyte differentiation literature, indicating a surprising fact that NFIB-induced astrocytes can reach the maturity level of adult astrocytes. Consequently, our results clearly show that NFIB-induced astrocytes already acquire physiological functions such as calcium responsiveness within a week, and this functional attribute further matures with additional differentiation for a week.

### 2) Glutamate Uptake Analysis

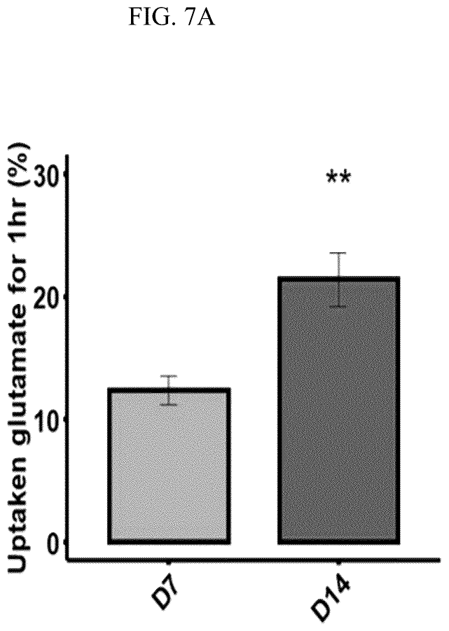

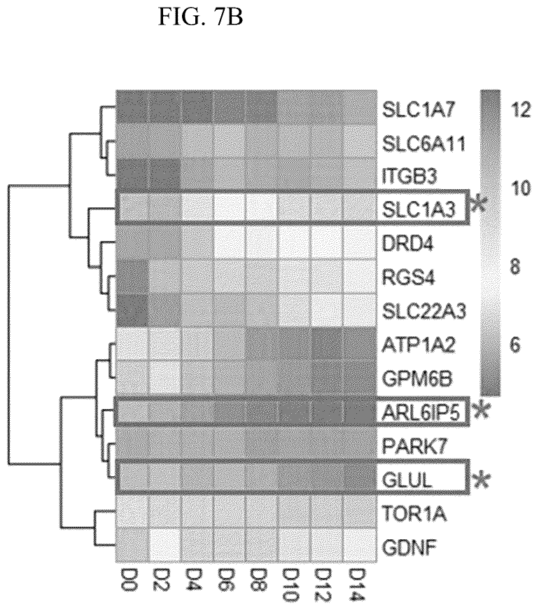

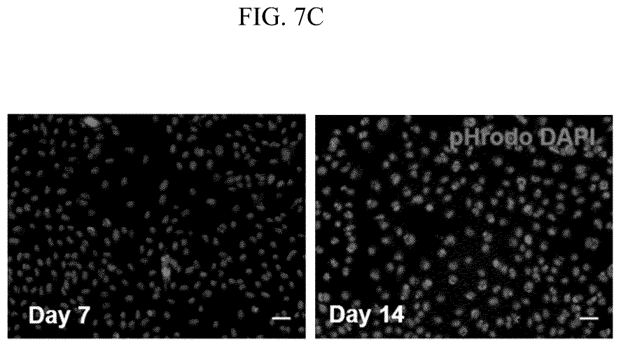

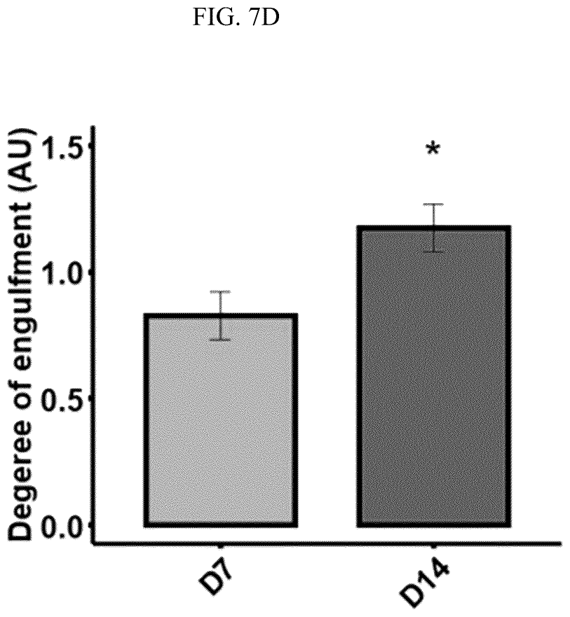

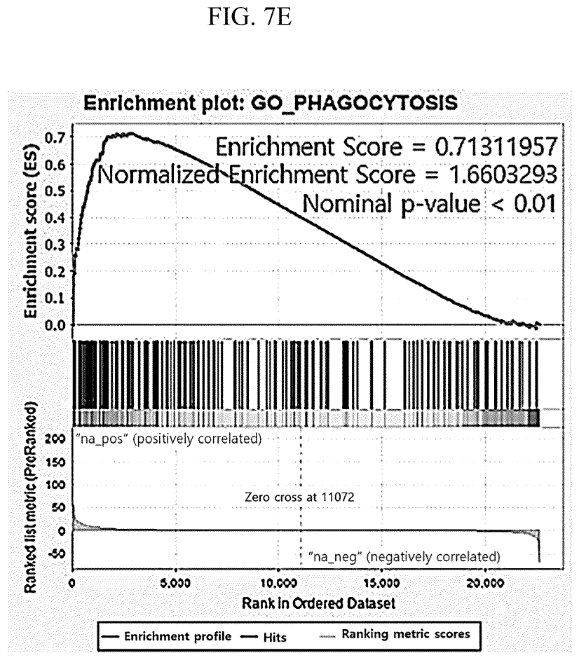

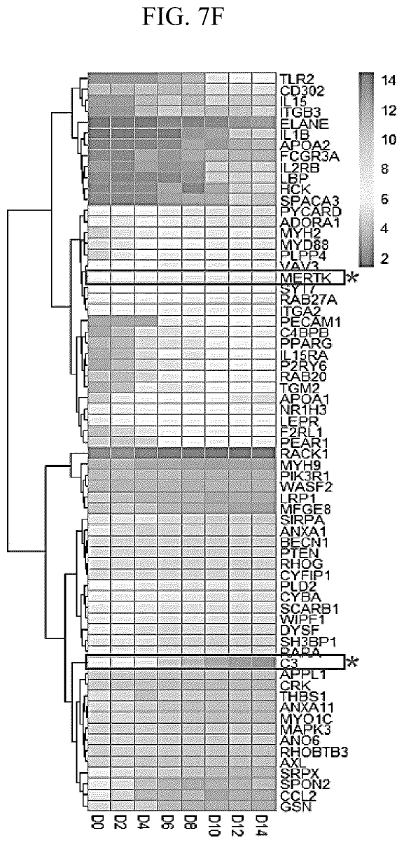

Uptake of excitatory neurotransmitters such as glutamate is another critical function of the astrocytes in the nervous system. This is because excessive glutamate concentration results in excitotoxic damage to the nervous tissue, potentially causing neurodegeneration. As there was a gradual upregulation of the neurotransmitter recycling protein, GLUL, in due course of gene expression (FIG. 5C, indicated by a red box with an asterisk), the ability of the NFIB-induced astrocytes to take up glutamate was assessed throughout differentiation to determine whether the ability improves. The astrocytes were exposed to 100 UM of glutamate, and the concentration of glutamate remaining in the medium was measured after 3 hours on day 7 and day 14. On day 14, the cells showed almost double (21.39±2.19%) the amount of glutamate uptake compared to an uptake of 12.34±1.17% on day 7 (p=0.007, n=6) (FIG. 7A). This increased uptake correlates well with the gradual increment of various genes related to neurotransmitter uptake (GO: 0001504), including SCL1A3 (a gene encoding excitatory amino acid transporter 1, also known as GLAST) and ARL6IP5 (a gene encoding glutamate transporter EEAC1-interacting protein). Thus, these results strongly suggest that the NFIB-induced astrocytes possess the ability to take up glutamate, which becomes gradually mature as the differentiation progresses.

### 3) Synaptosome Engulfment Assay

In the mammalian brain, astrocytes play a direct role in controlling the formation, maturation, and elimination of synapses. Particularly during development, astrocytes eliminate redundant synaptic connections through phagocytosis. This attribute of astrocytes is essential for the maturation of neural circuits and is closely related to the onset of neuropsychiatric disorders. To assess the ability of the NFIB-induced astrocytes to clear synapses, a synaptosome engulfment assay was performed. For this purpose, synaptosomes were isolated from the mouse hippocampi tissues at postnatal day 1 with Syn-PER® Synaptic Protein Extraction Reagent (Thermo Fisher Scientific) following the manufacturer's instructions. The isolated synaptosomes were conjugated with pHrodo-Red using pHrodo-Red Microscale Labeling Kit (Thermo Fisher Scientific) and then exposed to astrocytes on days 7 and 14 during NFIB-induced differentiation for 24 hours. The following day, at least five images per well were captured from random areas of the 6-well plates, and the degree of engulfment was calculated by measuring the area of synaptosomes (fluorescent signal) normalized to the cell counts. In this case, a pH-sensitive fluorescent dextran present in pHrodo-Red emits red fluorescence only when exposed to an acidic environment, such as in phagosome. This occurs only when it enters the cell through phagocytosis, rather than simply binding to the cell surface.

This assay revealed that on day 14, the cells engulfed significantly more synaptosomes and emitted greater red fluorescence than those on day 7 (1.17±0.21 and 0.83±0.21 in the arbitrary units, respectively) (FIG. 7C and FIG. 7D). GSEA analysis between day 0 and day 14 of differentiation showed increased expression of genes related to phagocytosis (GO:0006909), proving that the corresponding function is maturing at the molecular levels (FIG. 7E). Interestingly, it has been reported that among those upregulated genes, a few are expressed only in the postnatal and adult brain, but not in fetal ones. They include the proto-oncogene, tyrosine-protein kinase MER (MERTK), critically involved in synapse elimination, and a complement protein, C3, related to synaptic pruning. These results indicate that NFIB-induced astrocytes are capable of synaptosome phagocytosis within 7 days and induce more cells to attain maturity, acquiring the phagocytic activity at the later stages of differentiation.

Collectively, these functional analyses demonstrate that NFIB-induced astrocytes undergo functional maturation correlated with a transition in transcriptomic profiles from fetal to adult astrocytes within a short period.

### 1-6. Confirmation of the Potential as a Platform for Research on Human Astrocyte Development

Data thus far has shown that the astrocyte differentiation method involving the introduction of NFIB accurately replicates gene expression changes and functional maturation observed in in vivo astrogliogenesis. Given that the actual astrocyte differentiation, which typically spans over a year, is condensed to two weeks, and simultaneously, molecular/physiological changes closely resembling the authentic astrocyte differentiation process are faithfully simulated, the current differentiation method may serve as a valuable platform for studying the molecular mechanisms essential for astrocyte development.

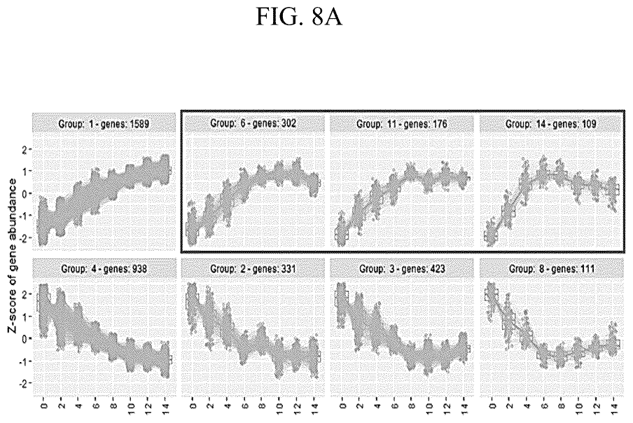

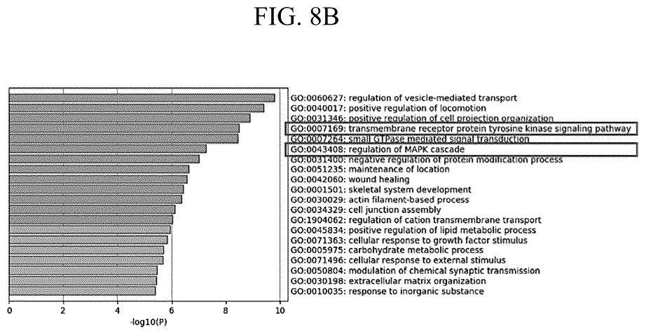

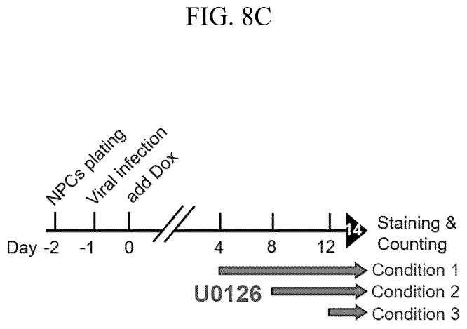

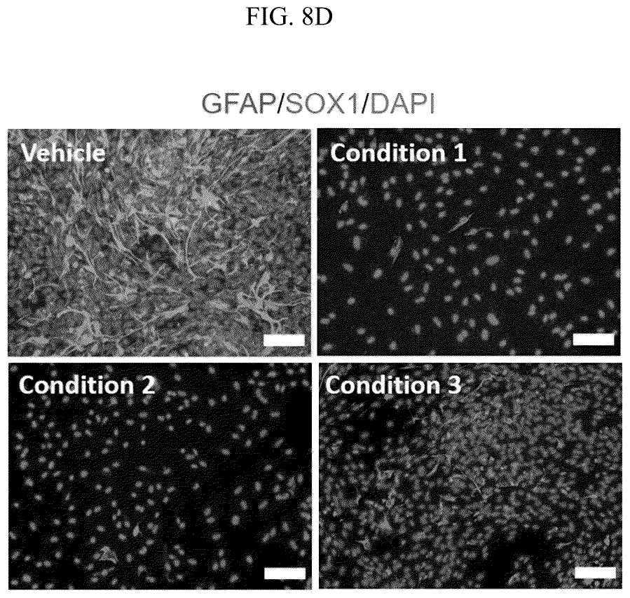

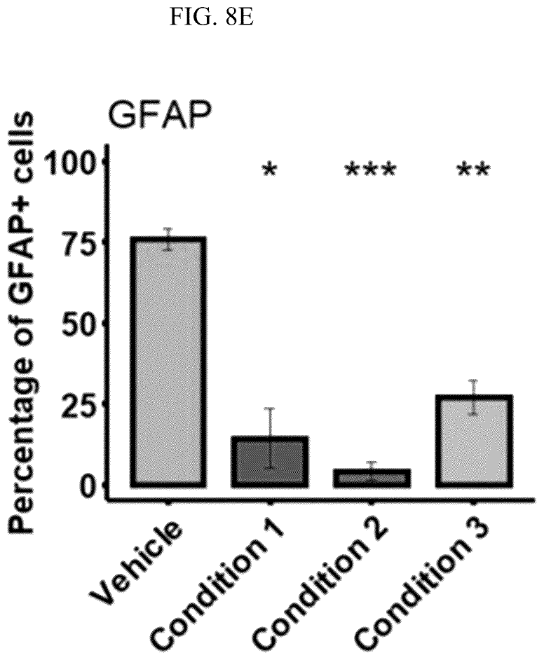

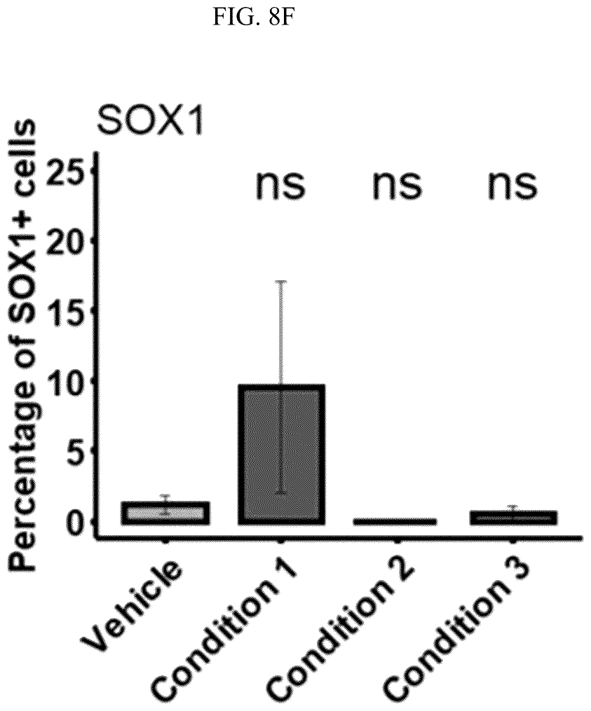

To further validate this notion, the gene clusters that underwent significant changes during the differentiation process were reexamined. The RNA-seq data was reassessed, and differentially expressed genes (DEGs) showing significant changes during the differentiation process, determined through a likelihood ratio test (p_{adj}<0.05), were primarily sorted, and categorized. This analysis is primarily used to identify gene clusters exhibiting a specific expression pattern along any biological change process. Of the eight groups categorized through this analysis, three groups exhibited expression dynamics similar to those of GFAP and ALDH1L1, which are representative astrocyte markers (FIG. 5A and FIG. 8A). Interestingly, the GO term analysis of the three groups revealed the significant enrichment of genes related to the receptors for protein kinase signaling pathways and the regulation of the MAPK cascade (FIG. 8B).

To confirm whether the MAPK signaling mechanism indeed plays an important role in astrocyte differentiation, cells were treated with U0126, an inhibitor of MEK1/2 (an upstream component of the MAPK signaling pathway), at a concentration of 20 μM during various time windows (FIG. 8C). In the present experiment, the treatment with U0126 at a concentration of 20 μM did not induce significant cell death but tended to slightly decrease cell proliferation. However, as illustrated in FIG. 8D and FIG. 8E, immunofluorescence staining followed by quantitative analysis revealed that inhibition of the MAPK signaling pathway with prolonged U0126 treatment (Treatment Condition 1 or 2) significantly reduced the number of GFAP-positive cells, although a short exposure to U0126 (Treatment Condition 3: treatment for two days from day 12 to day 14 of differentiation) still generated a substantial number of astrocytes (26.88% in total cells). Unlike the cells in the vehicle-treated group, the GFAP-positive cells generated under the influence of U0126 generally displayed a small cell body with fewer short processes, and this morphological change was correlated with the U0126 exposure time (FIG. 8D). The number of SOX1-positive cells was not significantly different among the groups, indicating that most cells were not undifferentiated (FIG. 8D and FIG. 8F). This data suggests that active MAPK signaling is required for astrocyte differentiation.

### Discussion

In the present invention, the inventors demonstrated that the overexpression of NFIB in hPSC-NPCs efficiently generates functional astrocytes in 2 weeks. Comparative transcriptome analysis revealed that NFIB-induced astrocytes exhibited a gene expression pattern resembling those of previously reported human fetal astrocytes and hPSC-derived astrocytes from previous studies. NFIB overexpression instantly initiated a gene expression program biased toward the astrocyte fate, gradually decreasing the expression of the genes of fetal astrocytes while increasing the expression of genes related to mature astrocytes. The expression kinetics of astrocyte-specific genes differentiated by the present method closely resembled those of astrocytes undergoing slow differentiation over about 590 days in 3D cortical cerebral spheroids. Furthermore, a 2-week differentiation induced by NFIB overexpression demonstrated functional maturation in calcium transients, glutamate uptake, and the engulfment of the synaptosome. These results suggest that overexpression of NFIB induces rapid and robust astrocyte differentiation, accompanied by the progressive transcriptomic and functional changes that mimic the molecular and cellular program of astrogliogenesis.

NFIB is a member of the CAATT element-binding transcription factor family, which includes NFIA, NFIC, and NFIX, and plays critical roles in development and stem cell differentiation. The expression and potential function of NFIB in a developing embryo were first demonstrated nearly two decades ago. Since then, its role as a transcriptional regulator in the development of various tissues, including the brain, lungs, muscles, and melanocyte stem cells in hair follicles, has been characterized. NFIB's involvement in the development of the central nervous system (CNS) has garnered broad attention due to observed brain defects in rodent models with genetic inactivation. These defects include morphogenic abnormalities in the hippocampus and corpus callosum dysgenesis, highlighting NFIB's significance in CNS development. This evidence suggested that NFIB may not merely act as a local transcriptional modulator in the development of particular regions but may serve a key modulator involved in the development of various CNS regions. Subsequently, the expression of NFIA and NFIB was detected in a region of the spinal cord inducing GLAST (the ventricular zone) and was found to directly promote the onset of astrogliogenesis in both the chicken and mouse spinal cord. While previous studies have placed a greater emphasis on the role of NFIA in astrogliogenesis compared to NFIB, the results suggest that both factors contribute to astrogliogenesis to a similar extent. However, recent findings in Reference 13 indicate that both genes share similar biological functions and act additively or complementarily, rather than redundantly. This means that the two factors target the similar gene sets in the developing brain. Nonetheless, the exact functional difference between the two genes has not yet been clearly elucidated.

The astrocyte differentiation method presented in the present invention offers advantages over methods in the related art, such as Non-Patent Document 2, for several reasons. First, the present invention utilized hPSC-NPCs rather than undifferentiated hPSCs as starting cells. When comparing differentiation efficiencies after the same period (2 weeks) and considering only the number of cells positive for astrocyte markers, both the method using hPSCs as starting cells and the present method using hPSC-NPCs yield similar results. However, the ability of the 2-week-old astrocytes to uptake glutamate, when compared among the NFIB-induced astrocytes, appeared significantly greater in the results (about 21%) of the present invention than in the results (about 12 to 13%) of Non-Patent Document 2 (FIG. 7A). This difference is not likely due to technical factors measuring the glutamate uptake capacity, as the analysis follow the same method as the previous documents, except for the vendor of the calorimetric assay kit. Moreover, 2-week-old astrocytes differentiated by the present method exhibit an “all-or-none” response to glutamate, a phenomenon not reported before. Such differences in cell maturity are likely manifested by variation in the developmental stage of the starting cells. Therefore, it is a reasonable to infer that the expression of NFIB in hPSC-NPCs, whose developmental stage is more advanced, induces the faster generation of astrocytes that are quicker and more mature than those derived from hPSCs.

Secondly, the utilization of hPSC-NPCs with an advanced developmental stage was not a factor that shortened the differentiation process. This can be demonstrated by the following evidence, refuting the notion that this outcome could have easily predicted from existing knowledge. A comparison between the previous strategy using NFIA overexpression in hPSC-LTNSCs (Non-Patent Document 3) and the method presented in the present invention revealed that astrocytes are induced more rapidly in the present invention. In Non-Patent Document 3, it took about 8 weeks after overexpressing NFIA in hPSC-LTNSCs to obtain 60% or more of GFAP-positive cells, whereas in the present invention, it was achieved in just 2 weeks. Despite the difference in differentiation (2 weeks vs. 8 weeks), the transcriptomes of differentiated astrocytes from both groups were closely clustered. Furthermore, the transcriptomes of astrocytes differentiated by the present differentiation method cluster more closely with astrocytes primarily cultured in human adult brain tissue (FIG. 4D). Therefore, it is clearly demonstrated that simply using hPSC-NPCs, which are more developed than hPSCs, is not the sole reason why this strategy shortened the differentiation time.

Despite the use of hPSC-LTNSCs in the study of Non-Patent Document 3, the following can be inferred as a reason for the longer differentiation period compared to that presented in the current invention. Firstly, the astrocyte differentiation ability of NFIA is lower than that of NFIB. However, an even more critical factor is that the hPSC-LTNSCs used in Non-Patent Document 3 are cells cultured under specific conditions for long-term culture, termed “long-term human embryonic stem cell-derived neural stem cells (LT-hESNSCs or LTNSCs)”. Notably, these cells exhibit a much stronger tendency to differentiate toward neurons rather than astrocytes or oligodendrocytes (Reference 14). This “neurogenic” tendency of LTNSCs might hinder the progress of differentiation toward astrocytes by NFIA. Consequently, Non-Patent Document 3 would have required more time to achieve a higher percentage of GFAP-positive cells than the present study.

Finally, in comparison to previous literature suggesting a combination of two or more transcription factors (SOX9/NFIA), the present method required the overexpression of only a single factor, NFIB, to induce astrocytes. Although the lentivirus is an efficient and widely used gene delivery system, its random-integration into the host genome has been problematic. In particular, when lentiviral-introduced cells are used in modeling for genetic diseases or regenerative medicine, problems may arise. Therefore, the usage of a minimum gene set is practically more beneficial than using two or more. It was confirmed that the efficient induction of astrocytes with NFIB alone could be achieved because the present invention revealed that, unlike hPSCs, NPCs already expressed SOX9 at the substantial level. Furthermore, it was intriguing that NFIB overexpression induced a greater elevation of the endogenous expression level of SOX9. Therefore, the upregulation of SOX9 by NFIB overexpression confirmed in the present invention may suggest an interesting possibility that SOX9 and NFI may participate in a positive feedback loop, each modulating the other's expression.

The present invention not only provides a rapid and efficient protocol for generating functional astrocytes but also offers a platform for investigating the molecular mechanism(s) of human astrogliogenesis. Recent brain organoid technology has provided an exceptional model system for studying human development, demonstrating that the 3D cerebral cortical spheroids derived from hPSCs recapitulate the molecular and physiological aspects of human astrogliogenesis. However, despite its remarkable authenticity, the lengthy process (taking a year or more) and subsequent cell purification hinder its application. On the other hand, the method according to the present invention requires only 2 weeks to obtain a highly enriched population of astrocytes with transcriptomic and physiological changes during differentiation resembling those occurring in vivo. Most importantly, the present inventors were able to exploit this differentiation technique to identify that the MAPK signaling pathways are required for the acquisition of astrocyte fate. While this may not be surprising given the role of MAPK signaling as a key regulator in gliogenesis, this finding still has an impact because it provides compelling evidence that the differentiation method of the present invention may faithfully follow the developmental program of astrogliogenesis. Therefore, the present method may serve as an in vitro model system for studying the mechanism of human astrogliogenesis.

In conclusion, the overexpression of NFIB in hPSC-NPCs generates functional astrocytes at a high yield in a short period, recapitulating the key features of in vivo astrogliogenesis. Therefore, this system may serve as a promising cellular platform for studying the development of human astrocytes and neurological disorders caused by developmental defect or dysfunction of astrocytes, such as Alexander disease, Rett syndrome, and fragile X syndrome. This differentiation paradigm, combined with a technology generating patient-specific iPSCs, is rapid, efficient, and scalable and can be utilized for a method of treating intractable neurological diseases.

### REFERENCES

1: Chambers, S. M., Fasano, C. A., Papapetrou, E. P., Tomishima, M., Sadelain, M., & Studer, L. (2009). Highly efficient neural conversion of human ES and iPS cells by dual inhibition of SMAD signaling. Nat Biotechnol, 27(3), 275-280. doi: 10.1038/nbt. 1529
2: Tcw, J., Wang, M., Pimenova, A. A., Bowles, K. R., Hartley, B. J., Lacin, E., Brennand, K. J. (2017). An Efficient Platform for Astrocyte Differentiation from Human Induced Pluripotent Stem Cells. Stem Cell Reports. 9(2), 600-614.

doi: 10.1016/j.stemcr.2017.06.018
3: Zhang, Y., Sloan, S. A., Clarke, L. E., Caneda, C., Plaza, C. A., Blumenthal, P. D., Barres, B. A. (2016). Purification and Characterization of Progenitor and Mature Human Astrocytes Reveals Transcriptional and Functional Differences with Mouse. Neuron. 89(1), 37-53. doi: 10.1016/j.neuron.2015.11.013
4: Bushnell, B. (2014). BBMap: A Fast, Accurate, Splice-Aware Aligner. United States.
5: Sloan, S. A., Darmanis, S., Huber, N., Khan, T. A., Birey, F., Caneda, C., Pasca, S. P. (2017). Human Astrocyte Maturation Captured in 3D Cerebral Cortical Spheroids Derived from Pluripotent Stem Cells. Neuron, 95(4), 779-790 e776. doi: 10.1016/j.neuron.2017.07.035
6: Caiazzo, M., Giannelli, S., Valente, P., Lignani, G, Carissimo, A., Sessa, A., Broccoli, V. (2015). Direct conversion of fibroblasts into functional astrocytes by defined transcription factors. Stem Cell Reports. 4(1), 25-36. doi:10.1016/j.stemcr.2014.12.002
7: Li Y, Guo B, Yang R, Xiao Z, Gao X, Yu J, Li S, Luo Y. (2018). A novel long noncoding RNA Inc158 promotes the differentiation of mouse neural pre-cursor cells into oligodendrocytes by targeting nuclear factor-IB. Neuroreport. 29(13): 1121-1128. doi: 10.1097/WNR.0000000000001083.
8: Rolando C, Erni A, Grison A, Beattie R, Engler A, Gokhale P J, Milo M, Wegleiter T, Jessberger S, Taylor V. (2016). Multipotency of Adult Hippocampal NSCs In Vivo Is Restricted by Drosha/NFIB. Cell Stem Cell. 2016 Nov. 3; 19(5):653-662. doi: 10.1016/j.stem.2016.07.003.
9: Scott, C. E., Wynn, S. L., Sesay, A., Cruz, C., Cheung, M., Gomez Gaviro, M. V., Briscoe, J. (2010). SOX9 induces and maintains neural stem cells. Nat Neurosci, 13(10), 1181-1189. doi: 10.1038/nn.2646
10: Deneen, B., Ho, R., Lukaszewicz, A., Hochstim, C. J., Gronostajski, R. M., & Anderson, D. J. (2006). The transcription factor NFIA controls the onset of gliogenesis in the developing spinal cord. Neuron, 52(6), 953-968. doi: 10.1016/j.neuron.2006.11.019
11: Liu, Y., Han, S. S., Wu, Y., Tuohy, T. M., Xue, H., Cai, J., Rao, M. S. (2004). CD44 expression identifies astrocyte-restricted precursor cells. Dev Biol. 276(1), 31-46. doi:10.1016/j.ydbio.2004.08.018
12: Piper, M., Barry, G, Harvey, T. J., McLeay, R., Smith, A. G, Harris, L., Richards, L. J. (2014). NFIB-mediated repression of the epigenetic factor Ezh2 regulates cortical development. J Neurosci, 34(8), 2921-2930. doi: 10.1523/JNEUROSCI.2319-13.2014
13: Bunt, J., Osinski, J. M., Lim, J. W., Vidovic, D., Ye, Y., Zalucki, O., Piper, M. (2017). Combined allelic dosage of Nfia and Nfib regulates cortical development. Brain Neurosci Adv, 1, 2398212817739433. doi:10.1177/2398212817739433
14: Philipp Koch, Thoralf Opitz, Julius A. Steinbeck, Julia Ladewig, and Oliver Brustle. (2009). A rosette-type, self-renewing human ES cell-derived neural stem cell with potential for in vitro instruction and synaptic intergration. Proc Natl Acad Sci. 106(9), 3225-3230.

1. A composition for inducing differentiation from neural precursor cells into astrocytes comprising: an NFIB protein; or a gene encoding the same.

2. The composition of claim 1, wherein the gene encoding the NFIB protein is introduced into neural precursor cells by a vector.

3. The composition of claim 2, wherein the vector is selected from the group consisting of a plasmid vector, a cosmid vector, a viral vector and an episomal vector.

4. The composition of claim 1, wherein the neural precursor cells are derived from pluripotent stem cells.

5. The composition of claim 1, wherein the neural precursor cells endogenously express SOX9.

6. The composition of claim 5, wherein the endogenous expression of SOX9 in the neural precursor cells is upregulated by introducing NFIB.

7. The composition of claim 1, wherein the differentiation is performed within 3 weeks.

8. A method for inducing differentiation from neural precursor cells into astrocytes, the method comprising: introducing an NFIB protein; or a gene encoding the same into neural precursor cells.

9. The method of claim 8, wherein differentiating pluripotent stem cells into neural precursor cells is performed before introducing an NFIB protein or a gene encoding the same into the neural precursor cells.

10. The method of claim 8, wherein the gene encoding the NFIB protein is introduced into neural precursor cells by a vector.

11. The method of claim 10, wherein the vector is selected from the group consisting of a plasmid vector, a cosmid vector, a viral vector and an episomal vector.

12. The method of claim 8, wherein the neural precursor cells are differentiated from pluripotent stem cells.

13. The method of claim 8, wherein the neural precursor cells endogenously express SOX9.

14. The method of claim 13, wherein the endogenous expression of SOX9 in the neural precursor cells is upregulated by introducing NFIB.

15. The method of claim 8, wherein the differentiation is performed within 3 weeks.

16. The method of claim 15, wherein GFAP-positive cells account for 80% or more of all differentiated cells within 3 weeks of differentiation in the method.
